(19) United States
(12) Patent Application Publication
(10) Pub. No.: US 2023/0005139 A1
(43) Pub. Date: Jan. 5, 2023

(54) Fibrotic Cap Detection In Medical Images

(71) Applicant: LightLab Imaging, Inc., Westford, MA (US)

(72) Inventors: Justin Akira Blaber, Lowell, MA (US); Ajay Gopinath, Bedford, MA (US); Gregory Patrick Amis, Westford, MA (US); Kyle Savidge, Medford, MA (US)

(73) Assignee: LightLab Imaging, Inc., Westford, MA (US)

(21) Appl. No.: 17/854,994

(22) Filed: Jun. 30, 2022

Related U.S. Application Data
(60) Provisional application No. 63/217,527, filed on Jul. 1, 2021.

(51) Int. Cl. G06T 7/00; G06T 7/12; G16H 30/40; G16H 50/20; G06N 20/00

(52) U.S. Cl. CPC G06T 7/0012; G06T 7/12; G16H 30/40; G16H 50/20; G06N 20/00; G06T 2207/20081; G06T 2207/30101; G06T 2207/10101; G06T 2207/10132; A61B 8/5223

(57) ABSTRACT

Aspects of the disclosure provide for methods, systems, and apparatuses, including computer-readable storage media, for lipid detection by identifying fibrotic caps in medical images of blood vessels. A method includes receiving one or more input images of a blood vessel and processing the one or more input images using a machine learning model trained to identify locations of fibrotic caps in blood vessels. The machine learning model is trained using a plurality of training images each annotated with locations of one or more fibrotic caps. A method includes identifying and characterizing fibrotic caps of lipid pools based on differences in radial signal intensities measured at different locations of an input image. A system can generate one or more output images having segments that are visually annotated representing predicted locations of fibrotic caps covering lipidic plaques.

### CROSS-REFERENCE TO RELATED APPLICATIONS

This application claims the benefit of the filing date of United States Provisional Patent Application No. 63/217,527 filed Jul. 1, 2021, the disclosure of which is hereby incorporated herein by reference.

### BACKGROUND

Optical coherence tomography (OCT) is an imaging technique with widespread applications in ophthalmology, cardiology, gastroenterology, and other fields of medicine and scientific study. OCT can be used in conjunction with various other imaging technologies, such as intravascular ultrasound (IVUS), near-infrared spectroscopy (NIRS), angiography, fluoroscopy, and X-ray based imaging.

To perform imaging, an imaging probe can be mounted on a catheter and maneuvered through a point or region of interest, such as through a blood vessel of a patient. The imaging probe can return multiple image frames of a point of interest, which can be further processed or analyzed, for example to diagnose the patient with a medical condition, or as part of a scientific study. Normal arteries have a layered structure that includes intima, media, and adventitia. As a result of some medical conditions, such as atherosclerosis, the intima or other parts of the artery may contain plaque, which can be formed from different types of fiber, proteoglycans, lipid, or calcium.

Neural networks are machine learning models that include one or more layers of nonlinear operations to predict an output for a received input. In addition to an input layer and an output layer, some neural networks include one or more hidden layers. The output of each hidden layer can be input to another hidden layer or the output layer of the neural network. Each layer of the neural network can generate a respective output from a received input according to values for one or more model parameters for the layer. The model parameters can be weights or biases. Model parameter values and biases are determined through a training algorithm to cause the neural network to generate accurate output.

### BRIEF SUMMARY

Aspects of the disclosure provide for automatic detection and characterization of fibrotic caps adjacent to regions or pools of lipid depicted in blood vessel images. A system including one or more processors can receive images of a blood vessel annotated with segments representing fibrotic caps of pools of lipid around the imaged blood vessel. The system can process these images to further annotate segments representing portions of the image showing background, the lumen of the blood vessel, media, and/or calcium. From these processed images, the system can train one or more machine learning models to identify one or more segments of fibrotic caps depicted in an input image of a blood vessel, which are indicative of pools of lipid in tissue surrounding the imaged blood vessel.

In addition, or alternatively, the system can detect and characterize fibrotic caps of pools of lipid based on measuring the decay rate of imaging signal intensity through the edge of an imaged lumen and into the surrounding tissue. Based on comparing the rate of decay with known samples, the system can predict the locations of fibrotic caps of lipid pools, as well as estimate characteristics of a fibrotic cap. Example characteristics can include such as its thickness and/or the boundary between the fibrotic cap and a lipid pool.

Aspects of the disclosure provide for methods, systems, and apparatuses, including computer-readable storage media, for lipid detection by identifying fibrotic caps in medical images of blood vessels. A method includes receiving one or more input images of a blood vessel and processing the one or more input images using a machine learning model trained to identify locations of fibrotic caps in blood vessels. The machine learning model is trained using a plurality of training images each annotated with locations of one or more fibrotic caps. A method includes identifying and characterizing fibrotic caps of lipid pools based on differences in radial signal intensities measured at different locations of an input image. A system can generate one or more output images having segments that are visually annotated representing predicted locations of fibrotic caps.

The fibrotic caps can be over lipidic plaques. Aspects of the disclosure provide for identifying fibrotic caps to identify the underlying lipidic plaques.

An aspect of the disclosure includes a method for fibrotic cap identification in blood vessels, the method including: receiving, by one or more processors, one or more input images of a blood vessel; processing, by the one or more processors, the one or more input images using a machine learning model trained to identify locations of fibrotic caps in blood vessels, wherein the machine learning model is trained using a plurality of training images annotated with one or more locations of one or more fibrotic caps, each fibrotic cap adjacent to a respective pool of lipid; and receiving, by the one or more processors and as output from the machine learning model, one or more output images having segments that are visually annotated representing predicted locations of fibrotic caps.

An aspect of the disclosure includes a system including: one or more processors configured to: receive one or more input images of a blood vessel; process the one or more input images using a machine learning model trained to identify locations of fibrotic caps in blood vessels, wherein the machine learning model is trained using a plurality of training images annotated with locations of one or more fibrotic caps, each fibrotic cap adjacent to a respective pool of lipid; and receive, as output from the machine learning model, one or more output images having segments that are visually annotated to represent or illustrate predicted locations of fibrotic caps.

An aspect of the disclosure includes one or more non-transitory computer-readable media storing instructions that when executed by one or more processors causes the one or more processors to perform operations including: receiving one or more input images of a blood vessel; processing the one or more input images using a machine learning model trained to identify locations of fibrotic caps in blood vessels, wherein the machine learning model is trained using a plurality of training images each annotated with locations of one or more fibrotic caps, each fibrotic cap adjacent to a respective pool of lipid; and receiving, as output from the machine learning model, one or more output images having segments that are visually annotated representing predicted locations of fibrotic caps.

An aspect of the disclosure provides for a method for fibrotic cap identification in blood vessels, the method including: receiving, by one or more processors, one or more input images of a blood vessel; processing, by the one or more processors, the one or more input images using a machine learning model trained to identify locations of fibrotic caps in blood vessels, wherein the machine learning model is trained using a plurality of training images annotated with one or more locations of one or more fibrotic caps, each fibrotic cap adjacent to a respective pool of lipid; receiving, by the one or more processors and as output from the machine learning model, one or more output images having segments that are visually annotated representing predicted locations of fibrotic caps; generating, using the one or more processors and from the one or more output images, an updated boundary of a fibrotic cap relative to an adjacent pool of lipid based on signal intensities for a plurality of points in the one or more input images. The plurality of points can be along one or more arc-lines enclosing the fibrotic cap.

Generating the updated boundary can include measuring signal intensities for a plurality of points along one or more arc-lines enclosing the fibrotic cap; and determining, based on a comparison of a measured rate of decay of the signal intensities for the plurality of points and a predetermined rate of decay of signal intensity through fibrotic caps of lipid, a boundary between the fibrotic cap and the adjacent pool of lipid. The signal intensities may be stored as metadata including a profile for the signal intensities.

The method can further include identifying lipidic plaques based on the locations for the one or more fibrotic caps. Generating the updated boundary can include updating the boundary based on a radial intensity profile including the measured signal intensities and associated with the one or more input images.

Other aspects of the foregoing include a system including one or more processors configured to perform a method for fibrotic cap detection. Other aspects of the foregoing include one or more computer-readable storage media, storing instructions that when executed by one or more processors, cause the one or more processors to perform a method for fibrotic cap detection.

An aspect of the disclosure provides for a method for fibrotic cap identification in blood vessels, the method including: receiving, by one or more processors, one or more input images of a blood vessel; generating, using the one or more processors, one or more first output images comprising a boundary of a fibrotic cap relative to an adjacent pool of lipid, the generating based on signal intensities for a plurality of points in the one or more input images; and processing, by the one or more processors, the one or more input images using a machine learning model trained to identify locations of fibrotic caps in blood vessels, wherein the machine learning model is trained using a plurality of training images annotated with one or more locations of one or more fibrotic caps, each fibrotic cap adjacent to a respective pool of lipid; receiving, by the one or more processors and as output from the machine learning model, one or more second output images having segments that are visually annotated representing predicted locations of fibrotic caps; and updating the boundary of the fibrotic cap in the one or more first output images using the one or more second output images. The plurality of points can be along one or more arc-lines enclosing the fibrotic cap.

An aspect of the disclosure provides for a method for fibrotic cap identification in blood vessels, the method including: receiving, by one or more processors, one or more input images of a blood vessel; generating, using the one or more processors, one or more first output images comprising a boundary of a fibrotic cap relative to an adjacent pool of lipid, the generating based on signal intensities for a plurality of points in the one or more input images; processing, by the one or more processors, the one or more first output images using a machine learning model trained to identify locations of fibrotic caps in blood vessels, wherein the machine learning model is trained using a plurality of training images annotated with one or more locations of one or more fibrotic caps, each fibrotic cap adjacent to a respective pool of lipid; receiving, by the one or more processors and as output from the machine learning model, one or more updated output images having segments that are visually annotated representing predicted locations of fibrotic caps.

The method may further include identifying one or more lipidic plaques based on the location of the identified one or more fibrotic caps. The method may further include saving the measured signal intensities as a profile in metadata corresponding to the one or more output images. The one or more updated output images can be updated using the profile of measured signal intensities, wherein the updating includes modifying boundaries for the one or more fibrotic caps generated using the machine learning model.

The foregoing and other aspects of the disclosure can include one or more of the following features. In some implementations, an aspect of the disclosure can include all of the following features in combination.

The one or more input images can be further annotated with segments corresponding to locations of at least one of calcium, the lumen in the blood vessel, or media.

The one or more input images include annotated segments representing one or more regions of media; wherein the one or more input images are images received from an imaging probe during a pullback of the imaging probe in the blood vessel; and wherein the method or operations further include: estimating, by the one or more processors, the average signal-to-noise ratio (SNR) of the one or more input images based on comparisons of predicted annotations of regions of media in the one or more input images and one or more ground-truth annotations of regions of media in the one or more input images; and in response, flagging, by the one or more processors, the one or more output images corresponding to the one or more input images in response to determining that the average SNR falls below a predetermined threshold.

The imaging probe can be an optical coherence tomography (OCT) imaging probe, an intravascular ultrasound (IVUS) imaging probe, a near-infrared spectroscopy (NIRS) imaging probe, an OCT-NIRS imaging probe, a micro-OCT (μOCT) imaging probe, etc. In some examples, the imaging probe may be configured to generate images according to a combination of the foregoing and other imaging techniques.

The imaging probe can be an optical coherence tomography (OCT) imaging probe.

Receiving the one or more output images can include receiving, for each input image, a respective visually annotated segment of the input image representing a predicted location for a fibrotic cap.

The method or operations can further include receiving, by the one or more processors and for each of the one or more output images, one or more measures of thickness for each fibrotic cap whose location is predicted in the output image.

The method or operations can further include generating, using the one or processors and from the one or more output images, an updated boundary of a fibrotic cap relative to an adjacent pool of lipid, wherein the generating includes: measuring, by the one or more processors, signal intensities for a plurality of points in the one or more input images; and determining, by the one or more processors and based on a comparison of a measured rate of decay of the signal intensities for the plurality of points and a predetermined rate of decay of signal intensity through fibrotic caps of lipid, a boundary between the fibrotic cap and the adjacent pool of lipid or lipidic plaque. The plurality of points can be along one or more arc-lines enclosing the fibrotic cap.

Determining the boundary between the fibrotic cap and the adjacent pool of lipid can include identifying a point of the plurality of points having a measured signal intensity that is proportional within a predetermined threshold to a peak signal intensity of the plurality of points.

The system can further include an imaging probe communicatively connected to the one or more processors; and receiving the one or more input images of the blood vessel can include receiving image data corresponding to the one or more input images from the imaging probe while the imaging probe is inside the blood vessel.

The system can further include one or more display devices configured for displaying image data; and wherein the one or more processors are further configured to display the one or more output images on the one or more display devices.

An aspect of the disclosure includes a method for training a machine learning model for fibrotic cap identification in blood vessels, the method including: receiving, by one or more processors, a plurality of training images, wherein each training image is annotated with one or more locations of one or more fibrotic caps in the training image, each fibrotic cap adjacent to a respective pool of lipid; processing, by the one or processors, the plurality of training images to annotate each training image with locations of at least one of calcium, a lumen in a blood vessel, or media; and training, by the one or more processors, the machine learning model using the processed plurality of training images. Processing the plurality of training images further includes processing the plurality of training images through one or more machine learning models trained to identify segments of input images that correspond to locations of at least one of calcium, the lumen in the blood vessel, and media.

An aspect of the disclosure provides for a system including one or more processors configured to receive a plurality of training images, wherein each training image is annotated with locations of one or more fibrotic caps in the training image, each fibrotic cap adjacent to a respective pool of lipid; process the plurality of training images to annotate each training image with respective one or more segments corresponding to locations of at least one of calcium, a lumen in a blood vessel, or media; and train the machine learning model using the processed plurality of training images.

An aspect of the disclosure provides for one or more transitory or non-transitory computer-readable media storing instructions that when executed by one or more processors causes the one or more processors to perform operations including: receiving a plurality of training images, wherein each training image is annotated with locations of one or more fibrotic caps in the training image, each fibrotic cap adjacent to a respective pool of lipid; processing the plurality of training images to annotate each training image with respective one or more segments corresponding to locations of at least one of calcium, a lumen in a blood vessel, and media; and training the machine learning model using the processed plurality of training images.

The machine learning model can be a first machine learning model; and the method can further include: receiving a second machine learning model including a plurality of model parameter values and trained to identify segments of input images that correspond to locations of least one of calcium, the lumen in the blood vessel, and media in an image of the blood vessel, and wherein training the first machine learning model includes initializing the training with at least a portion of model parameter values from the second machine learning model.

The second machine learning model can be a convolutional neural network including a plurality of layers, the plurality of layers including an output layer and each layer including one or more respective model parameter values; and training the first machine learning model can further include: replacing the output layer of the second machine learning model with a new layer configured to (i) receive the input to the output layer of the second machine learning model, and (ii) to generate, as output, a segmentation map for an input image, the segmentation map including a plurality of channels, including a channel for identifying segments of the input image representing predicted locations of one or more fibrotic caps in the input image, and training the second machine learning model with the replaced output layer using the processed plurality of training images.

The plurality of channels of the segmentation map can further include one or more channels for identifying segments of the input image representing predicted locations of at least one of calcium, the lumen for the blood vessel, and media.

Training the first machine learning model with the replaced output layer can further include updating model parameter values only for the new layer of the first machine learning model.

Processing the plurality of training images can further include processing the plurality of training images using the second machine learning model.

Training the machine learning model can include training the machine learning model to output, from an image of the blood vessel, a visually annotated segment of the image representing a predicted location for a fibrotic cap.

Training the machine learning model can include training the machine learning model to output, from an image of a blood vessel, one or more measures of at least one of thickness and length for each fibrotic cap identified in the image.

The imaging probe can be an optical coherence tomography (OCT) imaging probe, an intravascular ultrasound (IVUS) imaging probe, a near-infrared spectroscopy (NIRS) imaging probe, an OCT-NIRS imaging probe, a micro-OCT (μOCT) imaging probe, etc. In some examples, the imaging probe can be configured for multi-modal imaging, for example imaging using a combination of OCT, NIRS, OCT-NIRS, μOCT, etc.

The plurality of training images are images taken using optical coherence tomography (OCT).

An aspect of the disclosure provides for a method including: receiving, by one or more processors, an input image of a blood vessel; calculating, by the one or more processors and for an arc-line relative to vessel reference point in the input image, a respective signal intensity of an imaging signal for each of a plurality of points in the input image; and identifying, by the one or more processors and from the respective signal intensities of the plurality of points relative to a reference point, a fibrotic cap adjacent to a pool of lipid depicted in the input image. The plurality of points can be along one or more arc-lines enclosing the fibrotic cap.

An aspect of the disclosure provides for a system including: one or more processors, wherein the one or more processors are configured to: receive an input image of a blood vessel; calculate, for an arc-line relative to vessel reference point in the input image, a respective signal intensity of an imaging signal for each of a plurality of points in the input image; and identify, from the respective signal intensities of the plurality of points relative to a reference point, a fibrotic cap adjacent to a pool of lipid depicted in the input image. The plurality of points can be along one or more arc-lines enclosing the fibrotic cap.

An aspect of the disclosure provides for one or more transitory or non-transitory computer-readable media storing instructions that when executed by one or more processors causes the one or more processors to perform operations including: receiving, by one or more processors, an input image of a blood vessel; calculating, by the one or more processors and for an arc-line relative to vessel reference point in the input image, a respective signal intensity of an imaging signal for each of a plurality of points in the input image; and identifying, by the one or more processors and from the respective signal intensities of the plurality of points relative to a reference point, a fibrotic cap adjacent to a pool of lipid depicted in the input image. The plurality of points can be along one or more arc-lines enclosing the fibrotic cap.

The foregoing and other aspects of the disclosure can include one or more of the following features.

The reference point can be the center of a lumen of the blood vessel.

The input image can be annotated with the one or more arc-lines corresponding to a fibrotic cap.

The input image can be annotated with a segment corresponding to the fibrotic cap, and wherein the identifying includes identifying an updated boundary between the fibrotic cap and a pool of lipid. The input image is annotated with a visual representation of a fibrotic cap depicted in the input image, and wherein the method or operations can further include identifying the one or more arc-lines relative to the reference point and based on a coverage angle characterizing the fibrotic cap.

The plurality of points can form a sequence of points increasing in distance relative to the center of the lumen, wherein the first point of the sequence is closest to the reference points and the last point of the sequence is farthest from the reference point, and identifying the updated boundary between the fibrotic cap and the pool of lipid can include: calculating a rate of decay in signal intensity in the respective signal intensities of two or more points in the sequence of points along the arc-line; determining that the calculated rate of decay is within a threshold value of a predetermined rate of decay of signal intensity through a fibrotic cap and a pool of lipid; and in response to determining that the calculated rate of decay is within the threshold values, identifying the segment of the input image between the two or more points as the fibrotic cap.

Calculating the rate of decay in signal intensity can further include calculating the rate of decay in signal intensity for respective signal intensities of arc-lines farther from the center of the lumen than the second point along the one or more arc-lines.

Identifying the segment of the input image as the fibrotic cap can further include annotating the boundary between the fibrotic cap and a pool of lipid adjacent to the fibrotic cap.

Determining that the calculated rate of decay is within the threshold value of a predetermined rate of decay includes measuring the error of fit between the calculated rate of decay and a curve at least partially including the predetermined rate of decay over points at the same distance relative to the center of the lumen as the two or more points along the one or more arc-lines.

The method or operations can further include: in response to determining that the calculated rate of decay is not within a threshold value of the predetermined rate of decay for a fibrotic cap of a pool of lipid, determining that the rate of decay is within a respective threshold value for one or more other predetermined rates of decay, each of the other predetermined rates of decay corresponding to a respective measured rate of decay for an imaging signal through a respective non-lipid region of plaque or media.

The method or operations can further include identifying a first point of the plurality of points as corresponding to the peak signal intensity relative to the respective signal intensities of the plurality of points; identifying a second point of the plurality of points as corresponding to a respective signal intensity equal to a threshold intensity relative to the peak signal intensity; and measuring the thickness of the fibrotic cap as the distance between the edge of the lumen of the imaged blood vessel and the second arc-line.

The threshold intensity relative to the peak signal intensity can be 80 percent.

The method or operations can further include comparing one or both of a measure of thickness of an identified fibrotic cap and a decay rate of the plurality of points along the one or more arc-lines for an input image frames against one or more predetermined thresholds; and flagging the input image if one or both of the measure of thickness and the decay rate are within the one or more predetermined thresholds.

The method or operations can further include displaying the input image annotated with the location of the fibrotic cap.

The input image can be taken using an optical coherence tomography (OCT) imaging probe, an intravascular ultrasound (IVUS) imaging probe, a near-infrared spectroscopy (NIRS) imaging probe, an OCT-NIRS imaging probe, a micro-OCT (μOCT) imaging probe, etc.

The plurality of training images are images taken using optical coherence tomography (OCT), intravascular ultrasound (IVUS), near-infrared spectroscopy (NIRS), OCT-NIRS, or micro-OCT (μOCT), etc.

The input image can be taken using optical coherence tomography (OCT).

An aspect of the disclosure provides for a system including: one or more processors, wherein the one or more processors are configured to: receive, by one or more processors, an input image of a blood vessel; process, the input image using a machine learning model trained to identify locations of fibrotic caps in blood vessels, wherein the machine learning model is trained using a plurality of training images annotated with locations of one or more fibrotic caps, each fibrotic cap adjacent to a respective pool of lipid; receive, by the one or more processors and as output from the machine learning model, an output image including a segment that is visually annotated representing a predicted location of a fibrotic cap, including a boundary between the fibrotic cap and a pool of lipid; identify one or more arc-lines corresponding to the segment in the output image, the one or more arc-lines originating from a reference point in the output image; and identify an updated boundary between the fibrotic cap and a pool of lipid based on differences in signal intensity in the output image measured across one or more points of the one or more arc-lines.

Other aspects of the disclosure include methods, apparatus, and non-transitory computer readable storage media storing instructions for one or more computer programs that when executed, cause one or more processors to perform the actions of the methods.

### BRIEF DESCRIPTION OF THE DRAWINGS

FIG. 1 is a block diagram of an example image segmentation system, according to aspects of the disclosure.

FIG. 2 shows an example of a labeled fibrotic cap depicted in an example image frame.

FIG. 3 is a flowchart of an example process for training a fibrotic detection model, according to aspects of the disclosure.

FIG. 4A shows an example input image and corresponding output image generated by the image segmentation system and expressed in polar coordinates, according to aspects of the disclosure.

FIG. 4B shows the example input image and corresponding output image of FIG. 4A expressed in Cartesian coordinates.

FIG. 5 is a flowchart of an example process for training a fibrotic cap detection model using a previously trained model, according to aspects of the disclosure.

FIG. 6A is a flowchart of an example process for detecting fibrotic caps of lipid pools in tissue surrounding blood vessels, according to aspects of the disclosure.

FIG. 6B is a flowchart of an example process for flagging output images of a fibrotic cap detection model with a low signal-to-noise ratio, according to aspects of the disclosure

FIG. 7A illustrates multiple arc-lines from the center of a lumen depicted in an image frame.

FIG. 7B is a flowchart of an example process for identifying fibrotic caps based on radial signal intensities for arc-lines measured from an input image of a blood vessel, according to aspects of the disclosure.

FIG. 8 is a flowchart of an example process of identifying fibrotic caps based on the rate of decay of radial signal intensities for arc-lines measured from an input image of a blood vessel.

FIG. 9 shows graphs of peak signal intensities and rates of decay through different tissues and plaques.

FIG. 10 is a flowchart of an example process for measuring a thickness of a fibrotic cap using the peak radial signal intensity in a sequence of measured arc-lines measured from an input image of a blood vessel, according to aspects of the disclosure.

FIG. 11 is a block diagram of an example computing environment implementing the image segmentation system, according to aspects of the disclosure.

### DETAILED DESCRIPTION

### Overview

Aspects of the disclosure provide for automatic detection of fibrotic caps of pools of lipid in images of blood vessels. An image of a blood vessel can be taken by an imaging probe, for example an imaging probe on an imaging device such as a catheter configured to be maneuvered through a blood vessel or other region of interest in the body of a patient. Segments of the image can then be analyzed and labeled as corresponding to different tissues or plaques. Lipid accumulation in imaged blood vessels is of particular interest, as the presence and characteristics of lipid in and around a blood vessel can be used, for example, as part of screening a patient for different cardiovascular diseases.

One problem with OCT-captured images is that pools of lipid or lipidic plaque, unlike other tissues or plaques like calcium or media, often are not imaged as clearly as other segments of an imaged blood vessel. One reason for this is because the imaging signal from an imaging probe decays as the signal propagates through a fibrotic cap and into an adjacent pool of lipid. On the other hand, other plaques, such as calcium, can be easier to identify at least because those types of plaque do not have physical characteristics like lipid that causes imaging signals to quickly decay.

As a result, lipid is difficult to discern and often requires manual review by trained experts to identify. Even so, experts often are unable to directly characterize lipid, for example by characterizing the depth or width of a pool in tissue surrounding a blood vessel, because the pool often shows up unclear in an OCT-captured image. Another challenge is properly identifying the boundary between the fibrotic cap and the lipid pool itself. The boundary is difficult to identify at least because measuring the thickness of the fibrotic cap requires knowing where the cap ends, and the lipid pool begins. As a result, hand-labeled images of lipid and fibrotic caps are often not accurate and are time-consuming to produce. Further, expert annotation can often be inconsistent from image-to-image and from different experts annotating the same images. These problems in accurately identifying lipid can occur for images taken according to other modalities, such as when the images are captured from an intravascular ultrasound (IVUS) imaging probe, a near-infrared spectroscopy (NIRS) imaging probe, an OCT-NIRS imaging probe, a micro-OCT (μOCT) imaging probe, or any other of a variety of probes or devices implementing any of a variety of different imaging technologies.

Aspects of the disclosure provide for techniques for identifying lipid pools surrounding an imaged blood vessel, by training a model to identify fibrotic caps. A fibrotic cap of a pool of lipid can refer to tissue that caps a pool of lipid in tissue surrounding a blood vessel. A system as described herein can predict fibrotic caps of pools of lipid in an input image, as well as predict the presence of other regions of interest, such as calcium or media.

A system as provided herein can augment training data labeled with fibrotic caps capping pools of lipid, with labels of other regions of interest. Because automatic and manual annotation of segments of interest such as calcium can generally be performed accurately and quickly relative to identifying lipid, the system can leverage existing labels for other regions of interest in an image to accurately identify fibrotic caps than without those additional labels. The system can be configured to predict one or more channels of regions of interest visible from an input image, which can be used to annotate an input image of a blood vessel. The additional channels can correspond to the lumen of the blood vessel, calcium, media, lipid, and/or background of the input image, as examples.

Further, the system as provided herein can leverage differences in physical characteristics of lipid and non-lipid regions of interest, such as by the rate of signal decay of an imaging probe through different regions of interest. By identifying and comparing a signal decay rate of a region relative to the distance from the lumen edge, the system can predict whether the region corresponds to a lipid or a non-lipid. The system can predict physical characteristics of a fibrotic cap, such as thickness of the fibrotic cap, by comparing the decay rate of the signal intensity of an imaging signal against previously received samples of signal intensity decay rate through different tissues and plaques.

Further, the system as provided herein can more accurately identify the boundary between a fibrotic cap and a lipid pool by measuring and comparing signal intensities of an OCT image along points of arc-lines for an arc containing a fibrotic cap or lipid pool. In some examples, the system can receive, as input, hand-annotated images or images annotated according to aspects of the disclosure provided herein, of one or more fibrotic caps. The system can annotate an image with the boundary between the fibrotic cap and an adjacent lipid pool. As part of receiving the input images annotated with fibrotic caps, the system can calculate one or more arc-lines based on the coverage angles of the annotated caps. In some implementations, the system can process images taken using NIRS, IVUS, OCT-NIRS, and/or μOCT, etc., to measure and to compare signal intensities of an image, identifying a fibrotic cap and/or a boundary between the cap and a lipid pool or lipid plaque.

In some examples, the system receives only images annotated with arc-lines corresponding to an angle containing a fibrotic cap and can identify the boundary between the fibrotic cap enclosed by the arc-lines, and an adjacent pool of lipid. In those examples, the system can be configured to perform signal intensity analysis for points along the arc-lines to predict the location of a fibrotic cap, including a boundary between cap and lipid pool.

Physical characteristics for the fibrotic cap can be used for improved diagnosis of coronary conditions, such as thin cap fibroatheroma (TCFA) in an imaged blood vessel. By improving the accuracy of measuring the thickness of a fibrotic cap over other approaches, the system can provide data that can be used with a higher measure of confidence in diagnosing patients. Accurate measurements can be particularly important when differences in thickness of a fibrotic cap can be the difference between diagnosing a patient between relatively benign thick-cap fibroatheroma versus more dangerous conditions, such as TCFA. In addition, the system described herein can identify a boundary based on an adjustable threshold, which can be adjusted according to a rubric or standard for diagnosing or evaluating fibrotic caps for cardiovascular disease or increased risk of plaque rupture, such as TCFA, or based on observations of previously analyzed samples of fibrotic caps, e.g., in OCT-captured images, IVUS-captured images, in NIRS-captured images, in OCT-NIRS-captured images, in μOCT-captured images and/or images captured using any one of a variety of different imaging technologies.

### Example Systems

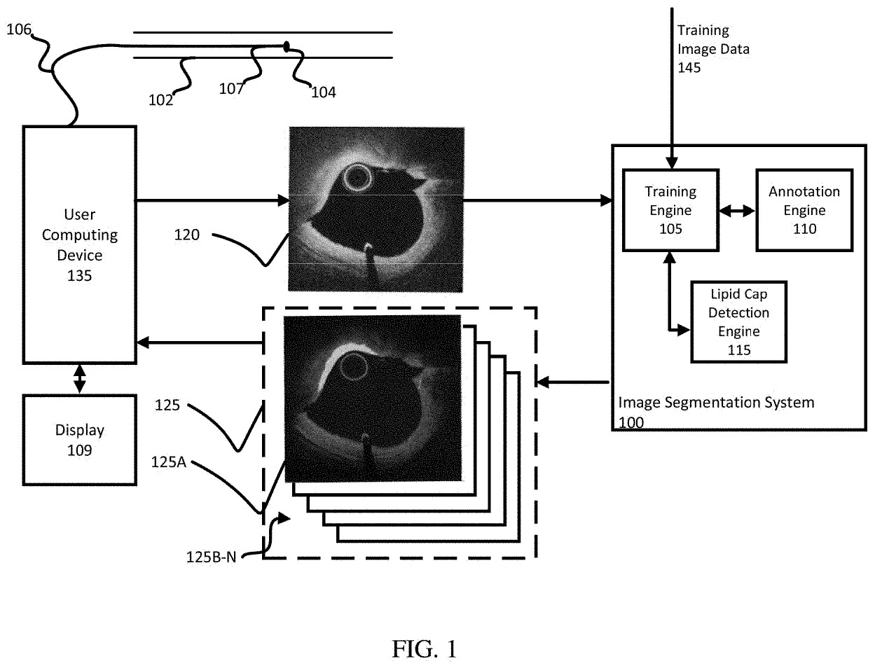

FIG. 1 is a block diagram of an image segmentation system 100, according to aspects of the disclosure. The image segmentation system 100 can include one or more processors and memory devices in one or more locations and across one or more devices, such as a server computing device or a computing device connected to imaging equipment and/or other tools, for example in a catheterization lab. The image segmentation system 100 can include a training engine 105, an annotation engine 110, and a fibrotic cap detection engine 115.

In general, the image segmentation system 100 is configured to receive input images, such as input image 120 of blood vessels, such as blood vessel 102, taken by an imaging device 107. The system 100 can generate, as output, one or more output images 125, including a fibrotic cap-annotated image 125A and optionally one or more annotated images 126B-N. The fibrotic cap-annotated image 125A can visually annotate segments of fibrotic caps in the input image 120 predicted by the system 100. For example, the fibrotic cap-annotated image 125A can include an overlay of highlighted or otherwise visually distinct portions of predicted fibrotic caps in the input image 120. From the fibrotic cap-annotated image 125A, the system can measure or estimate one or more physical characteristics of the identified cap, such as the thickness of the cap.

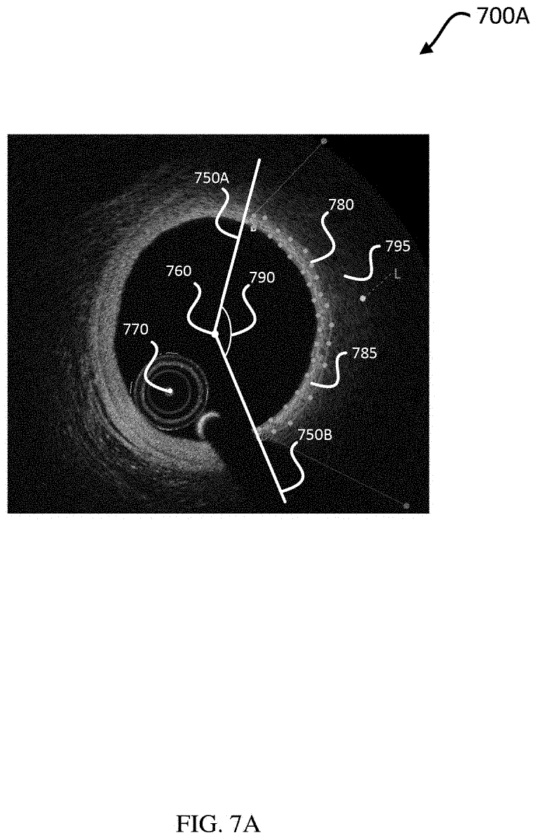

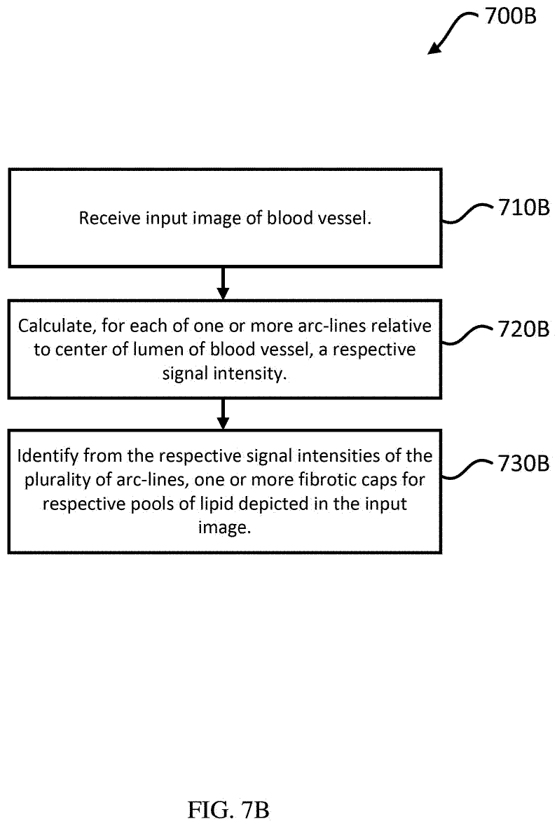

As described in more detail with reference to FIGS. 7A-10, the image segmentation system 100 can be configured to refine the boundary by analyzing the decay of the imaging signal measured from the input image 120, and by comparing a measured rate decay of the imaging signal through the cap, with previously obtained rates of decay for an imaging signal through various plaques, including lipid.

The system 100 can in some examples output other annotated images 125B-N representing predicted locations of other types of regions of interest, such as calcium, media, background, and the size and shape of the lumen of the blood vessel 102. The annotated images 125B-N can each be associated with a particular type of region of interest, such as calcium or media. In some examples, the system 100 generates a segmentation map or other data structure that maps pixels in the input image 120 to one or more channels, each channel corresponding to a region of interest.

A segmentation map can include multiple elements, for example elements in an array in which each element corresponds to a pixel in the input image 120. Each element of the array can include a different value, e.g., an integer value, where each value corresponds to a different channel for a predicted region of interest. For example, the output segmentation map can include elements with the value “1” for corresponding pixels predicted to be of a fibrotic cap. The output segmentation map can include other values for other regions of interest, such as the value “2” for corresponding pixels predicted to be of calcium. The system 100 can be configured to output segmentation maps with some or all of the channels represented in the map.

In some examples, instead of generating the output images 125, the system can generate the segmentation map of one or more channels, and the user computing device 135 can be configured to apply the segmentation map to the input image 120. As an example, the user computing device 135 can be configured to process the input image 120 with one or more channels of the segmentation map to generate corresponding images for the one or more channels, e.g., a fibrotic cap-annotated output image, a calcium-annotated output image, etc. Multiple channels can be combined, for example to generate an output image that is annotated for regions of calcium and lipid.

The prediction of different regions of interest, such as fibrotic caps for lipid pools, can be annotated in a number of different ways. For example, the segmentation map can be one or more masks of one or more channels that can be applied as overlays to the input image 120. As another example, the system 100 can copy and modify pixels of the input image 120 corresponding to locations for different predicted regions of interest. For example, the system 100 can generate the fibrotic cap-annotated image 125A having pixels of the input image 120 where fibrotic caps are predicted to be located and that are shaded or modified to appear visually distinct from the rest of the input image 120. In other examples, the system can modify pixels corresponding to a predicted fibrotic cap in other ways, such as through an outline of the predicted cap, visually distinct patterns, shading or thatching, etc.

The user computing device 135 can be configured to receive the input image 120 from an imaging device 107 having an imaging probe 104. The imaging probe 104 may be an OCT probe and/or an IVUS catheter, as examples. While the examples provided herein refer to an OCT probe, the use of an OCT probe or a particular OCT imaging technique is not intended to be limiting. For example, an IVUS catheter may be used in conjunction with or instead of the OCT probe. A guidewire, not shown, may be used to introduce the probe 104 into the blood vessel 102. The probe 104 may be introduced and pulled back along a length of the lumen of the blood vessel 102 while collecting data, for example as a sequence of image frames. According to some examples, the probe 104 may be held stationary during a pullback such that a plurality of scans of OCT and/or IVUS data sets may be collected. The data sets, which can include image frames or other image data, may be used to identify fibrotic caps for lipid pools and other regions of interest. According to some examples, the probe 104 can be configured for micro-optical coherence tomography (μOCT). Other imaging technologies may also be used, such as near-infrared spectroscopy and imaging (NIRS).

The probe 104 may be connected to the user computing device 135 through an optical fiber 106. The user computing device 135 may include a light source, such as a laser, an interferometer having a sample arm and a reference arm, various optical paths, a clock generator, photodiodes, and other OCT and/or IVUS components. In some examples, the user computing device 135 is connected to one or more other devices and/or pieces of equipment (not shown) configured for performing medical imaging using the imaging device 107. As an example, the user computing device 135 and the imaging device 107 can be part of a catheterization lab. In other examples, the system 100, the user computing device 135, display 109, and the imaging device 107 are part of a larger system for medical imaging, for example implemented as part of a catheterization lab.

The system 100 can be configured to receive and process the input image 120 in real-time, e.g., while the imaging device 107 is maneuvered while imaging the blood vessel 102. In other examples, the system 100 receives one or more image frames after a procedure is performed for imaging the blood vessel 102, for example by receiving input data that has been stored on the user computing device 135 or another device. In other examples, the system 100 receives input images from a different source altogether, for example from one or more devices over a network. In this latter example, the system 100 can be configured, for example, on one or more server computing devices configured to process incoming input images according to the techniques described herein.

As shown, the display 109 is separate from the user computing device 135, however, according to some examples, the display 109 may be part of the computing device 135. The display 109 may output image data such as the output images 125. The display 109 can display output in some examples through a display viewport, such as a circular display viewport.

The display 109 can show one or more image frames, for example as two-dimensional cross-sections of the blood vessel 102 and surrounding tissue. The display 109 can also include one or more other views to show different perspectives of the imaged blood vessel or another region of interest in the body of a patient. As an example, the display 109 can include a longitudinal view of the length of the blood vessel 102 from a start point to an end point. In some examples, the display 109 can highlight certain portions of the blood vessel 102 along the longitudinal view and can at least partially occlude other portions that are not currently selected for view. In some examples the display 109 is configured to receive input to scrub through different portions of the lumen 201 as shown in the longitudinal view.

The output can be displayed in real-time, for example during a procedure in which the imaging probe 104 is maneuvered through the blood vessel 102. Other data that can be output, for example in combination with the output images 125, include cross-sectional scan data, longitudinal scans, diameter graphs, lumen borders, plaque sizes, plaque circumference, visual indicia of plaque location, visual indicia of risk posed to stent expansion, flow rate, etc. The display 109 may identify features with text, arrows, color coding, highlighting, contour lines, or other suitable human or machine-readable indicia.

According to some examples the display 109 may be a graphic user interface (“GUI”). One or more steps of processes described herein may be performed automatically or without user input to navigate images, input information, select and/or interact with an input, etc. The display 109 alone or in combination with the user computing device 135 may allow for toggling between one or more viewing modes in response to user inputs. For example, a user may be able to toggle between different side branches on the display 109, such as by selecting a particular side branch and/or by selecting a view associated with the particular side branch.

In some examples, the display 109, alone or in combination with the user computing device 135, may include a menu. The menu may allow a user to show or hide various features. There may be more than one menu. For example, there may be a menu for selecting blood vessel features to display, such as to toggle on or off one or more masks for overlaying on top of the input image 120. Additionally, or alternatively, there may be a menu for selecting the virtual camera angle of the display. In some examples the display 109 can be configured to receive input. For example, the display 109 can include a touchscreen configured to receive touch input for interacting with a menu or other interactable element displayed on the display 109.

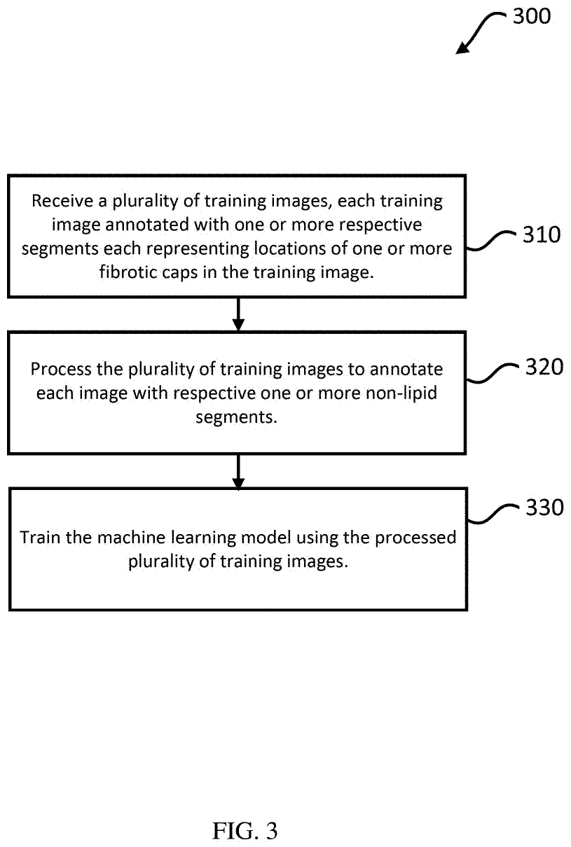

The output image frames 125 can be used, for example, as part of a downstream process for medical analysis, diagnosis, and/or general study. For example, the output image frames 125 can be displayed for review and analysis by a user, such as a medical professional, or used as input to an automatic process for medical diagnosis and evaluation, such as an expert system or other downstream process implemented on one or more computing devices, such as the one or more computing devices implementing system 100. From the annotated segments of the output image frames 125, the system 100 can estimate the thickness for one or more identified fibrotic caps adjoining respective pools of lipid in tissue around the blood vessel 102, for example according to techniques described herein with reference to FIGS. 3, and 7A-10.

For example, based on the estimated physical characteristics of predicted fibrotic caps, the output image frames 125 can be used at least partially for diagnosing TCFA and/or other coronary conditions. TCFA can be difficult to diagnose by visual inspection alone, at least because of the aforementioned physical characteristics of lipid pools in attenuating OCT or other imaging signals, such as in images taken using NIRS, OCT-NIRS, IVUS, μOCT, etc., which can make it difficult to ascertain the boundary between the fibrotic cap and its corresponding lipid pool.

Turning to the engines 105, 110, and 115, the fibrotic cap detection engine 115 is generally configured to predict, for example in the form of a highlight or other visible indicia, the presence of fibrotic caps in the input image 120. The fibrotic cap detection engine 115 can be configured to detect the fibrotic cap of a lipid pool, for example through one or more machine learning models trained as described herein with reference to FIGS. 3-6. In some examples, the fibrotic cap detection engine 115 can be configured to identify and characterize fibrotic caps by analyzing radial signal intensities of input images of blood vessels against known samples of fibrotic caps for lipid pools, as described herein with reference to FIGS. 7-10. In some implementations, the fibrotic cap detection engine 115 can be configured to identify and characterize fibrotic caps using a combination of the techniques described herein. Characterization of fibrotic caps or other regions of plaque in an input image can refer to the generation or measurement of quantitative or qualitative features of the cap or region of plaque. Example features that may form part of the characterization can include the length, width, or overall geometry of the cap or region of plaque.

The training engine 105 is configured to receive incoming training image data 145 and train one or more machine learning models implemented as part of the fibrotic cap detection engine 115. The training image data 145 can be image frames of blood vessels annotated with fibrotic caps. As described herein with reference to FIGS. 3-6B, the training engine 105 can be configured to train one or more machine learning models implemented as part of the fibrotic cap detection engine 115. The training engine 105 can use the training image data 145 annotated with the locations of fibrotic caps in corresponding image frames, and further annotated with the locations of other regions of interest by the annotation engine 110.

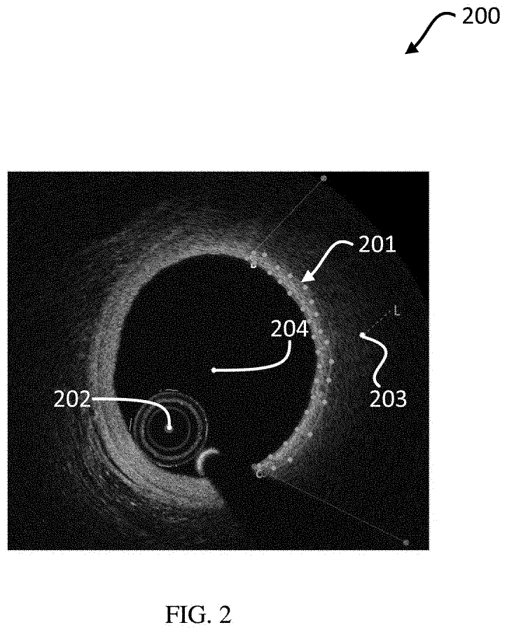

FIG. 2 shows an example of a labeled fibrotic cap 201 in an example image frame 200. In FIG. 2, the labeled fibrotic cap 201 is outlined by a series of points, although the fibrotic cap 201 can be represented in other ways, such as by shading the region of the cap relative to the rest of the image frame 200. The label can be generated, for example, by hand and according to a rubric for evaluating image frames for fibrotic caps. The image frame 200 can also be additionally labeled, for example with the location of the center of a lumen 204, the center 202 of an imaging device at the time the image frame was captured by the device, and region 203 indicating the lipid pool adjacent to the fibrotic cap 201.

In some examples, the training engine 105 receives image frames for training, such as the image frame 200 of FIG. 2 that are not annotated with one or more other regions of interest, such as calcium, media, or the outline or area covered by the lumen of an imaged blood vessel. The annotation engine 110 can be configured to receive the training image data 145 and further annotate image frames of the data with annotations corresponding to regions of calcium, media, a lumen, background, etc. To do so, the annotation engine 110 can be implemented using any of a variety of different techniques for identifying and classifying these regions of interest, for example using one or more appropriately trained machine learning models.

For example, the annotation engine 110 can implement one or more machine learning models trained to identify and classify regions of calcium depicted in an image frame. In some examples, the annotation engine 110 generates a modified image frame with a visual indication of predicted regions of interest, such as an outline surrounding a region of calcium or other identified non-lipid plaque. In other examples, the annotation engine 110 can generate data that the training engine 105 is configured to process in addition to a corresponding training image frame. The generated data can be, for example, data defining a mask over pixels of the training image frame, where each pixel of the training image frame corresponds to a pixel in the mask and indicates whether the pixel partially represents a region of interest depicted in the training image.

The generated data can, in some examples, include coordinate data corresponding to pixels of the processed image frame that at least partially depict a region of interest. For example, the generated data can include spatial or Cartesian coordinates for each pixel of a mask corresponding to a region of detected non-lipid plaque. The annotation engine can also be configured to optionally convert coordinate data according to one system (such as Cartesian coordinates) to another coordinate system (such as polar coordinates relative to a reference point, such as the center of a lumen).

Similarly, the training image data 145 can also include data defining the positions of pixels annotated as corresponding to regions of fibrotic caps of lipid pools, which the system 100 can be configured to convert depending on the input requirements for the training engine 105 and/or the fibrotic cap detection engine 115. One reason for the use of different coordinate systems can be because the training image data 145 is hand-labeled with multiple individual points that collectively define a perimeter for an annotated fibrotic cap, but the fibrotic cap detection engine 115 is configured to process the same image data with locations for different points expressed in polar coordinates. The system 100 can be configured to generate the output images 125 with the positions of pixels arranged according to a polar coordinate system, and optionally convert the output images 125 back to Cartesian coordinates for display on the display 109.

Although the training engine 105 is shown as part of the image segmentation system 100, in some examples the training engine 105 is implemented on one or more devices different from one or more devices implementing the rest of the system 100. Further, the system 100 may or may not train or fine-tune the one or more machine learning models described, and instead receive models pre-trained according to aspects of the disclosure.

### Example Methods

FIG. 3 is a flowchart of an example process 300 for training a fibrotic cap detection model, according to aspects of the disclosure. The process 300 can be performed by a system including one or more processors, located in one or more locations, and appropriately configured according to aspects of the disclosure.

The fibrotic cap detection model can include one or more machine learning models, such as neural networks, which can be trained using labeled image training data, for example the training image data 140 as described with reference to FIG. 1. A fibrotic cap detection engine, such as the fibrotic cap detection engine 115 of the system 100, can implement one or more mathematical models, e.g., the one or more machine learning models described above with reference to FIGS. 1-2, collectively referred to as the fibrotic cap detection model, and be configured to identify and characterize fibrotic caps of lipid pools of image frames as described herein.

The system receives a plurality of training images, each training image annotated with the locations of one or more fibrotic caps in the training image, according to block 310. As described herein with reference to FIGS. 1-2, the system can receive training image data labeled with locations of fibrotic caps detected in each image. As part of receiving the plurality of training images, the system can split the data into multiple sets, such as image frames for training, testing, and validation.

The system processes the plurality of training images to annotate each image with one or more non-lipid segments of the input image, according to block 320. For example, and as described with reference to FIG. 1, the system can include an annotation engine configured to annotate training image frames with annotations identifying predicted regions of non-lipid plaque, such as calcium, media, the lumen of the blood vessel, and background.

The system trains the fibrotic cap detection model using the processed plurality of training images, according to block 330. In some examples, the model is configured to generate, as output, a segmentation map representing predicted one or more regions of interest, including portions of the input image predicted to be fibrotic caps.

The fibrotic cap detection model can be trained according to any technique for supervised learning, and in general training techniques for machine learning models using datasets in which at least some of the training examples are labeled. For example, the fibrotic cap detection model can be one or more neural networks with model parameter values that are updated as part of a training process using backpropagation with gradient descent, either on individual image frames or on batches of image frames, as examples.

In some examples, the fibrotic cap detection model can be one or more convolutional neural networks configured to receive, as input, pixels corresponding to an input image, and generate, as output, a segmentation map corresponding to one or more channels of regions of interest in the input image. As an example, the fibrotic cap detection model can be a neural network including an input layer and an output layer, as well as one or more hidden layers in between the input and output layer. Each layer can include one or more model parameter values. Each layer can receive one or more inputs, such as from a previous layer in the case of a hidden layer, or a network input such as an input image in the case of the input layer. Each layer receiving an input can process the input through one or more activation functions that are weighted and/or biased according to model parameter values for the layer. The layer can pass output to a subsequent layer in the network, or in the case of the output layer, be configured to output a segmentation map and/or one or more output images, for example as described herein with reference to FIGS. 1-2.

The fibrotic cap detection model as a neural network can include a variety of different types of layers, such as pooling layers, convolutional layers, and fully connected layers, which can be arranged and sized according to any of a variety of different configurations for receiving and processing input image data.

Although examples of the fibrotic cap detection model are provided as neural networks and convolutional neural networks, a machine learning model can refer to any model or system configured to receive input and generate output according to the input and that can be trained to generate accurate output using input training image data and/or data extracted from the input training image data. If the fibrotic cap detection model includes more than one machine learning model, then the machine learning models can be trained and executed end-to-end, such that output for one model can be input for a subsequent model until reaching an output for a final model.

In some examples, the fibrotic cap detection model at least partially includes an encoder-decoder architecture with skip connections. As another example, the fibrotic cap detection model can include one or more neural network layers as part of an autoencoder trained to learn compact (encoded) representations of images from unlabeled training data, such as images taken using OCT, IVUS, NIRS, OCT-NIRS, μOCT, or taken using any other of a variety of other imaging technologies. The neural network layers can be further trained on input training images as described herein, and the fibrotic cap detection model can benefit from a broader set of training by being at least partially trained using unlabeled training images.

In some implementations, the fibrotic cap detection model can be trained to predict arc-lines for coverage angles containing one or more lipid caps depicted in an input image. The fibrotic cap detection model can generate output images annotated with arc-lines, which can be used as input for refining or identifying the boundary of a lipid cap and adjacent lipid pool, as described herein with reference to FIGS. 7A-10.

In some implementations, the fibrotic cap detection model can be trained for lipid cap classification in addition to or as an alternative to image segmentation as described herein. In those implementations, the fibrotic cap detection model can be trained with the same input training image data, annotated with locations of lipid caps, as well as non-lipid regions of interest.

The training can be done using a loss function that quantifies the difference between a location predicted by the system for a fibrotic cap of a lipid pool, versus the ground-truth location for the fibrotic cap received as part of the training data for the input image.

The loss function can be, for example a distance between the predicted location and ground-truth location for a fibrotic cap, measured at one or more pairs of points on the predicted and ground-truth location. In general, any loss function that compares each pixel between a training image with a predicted annotation and a training image with a ground-truth annotation can be used. Example loss functions can include computing a Jaccard similarity coefficient or score between the training image frame with the predicted location of the fibrotic cap and the training image frame with the ground-truth location for the fibrotic cap. Another example loss function can be a pixel-wise cross entropy loss, although any loss function used for training models for performing image segmentation tasks can be applied.

The cost function used as part of training the system can be an average of the values of the loss function values over at least a portion of the processed training image frames. For example, the cost function can be the mean Jaccard score over a set of training image frames. The system can train the fibrotic cap detection model until meeting one or more training criteria, which can specify, for example, a maximum error threshold when processing a validation test set of training images. Other training criteria can include a minimum or maximum number of training iterations, e.g., measured as a number of epochs (i.e., complete passes over all of the training data), batches and/or individual training examples processed, or a minimum or maximum amount of time spent executing the process 300 for training the fibrotic cap detection model.

The system can perform training until determining that one or more stopping criteria have been met. For example, the stopping criteria can be a preset number of epochs, a minimum improvement of the system between epochs as measured using the loss function, the passing of a predetermined amount of wall-clock time, and/or until a computational budget is exhausted, e.g., a predetermined number of processing cycles.

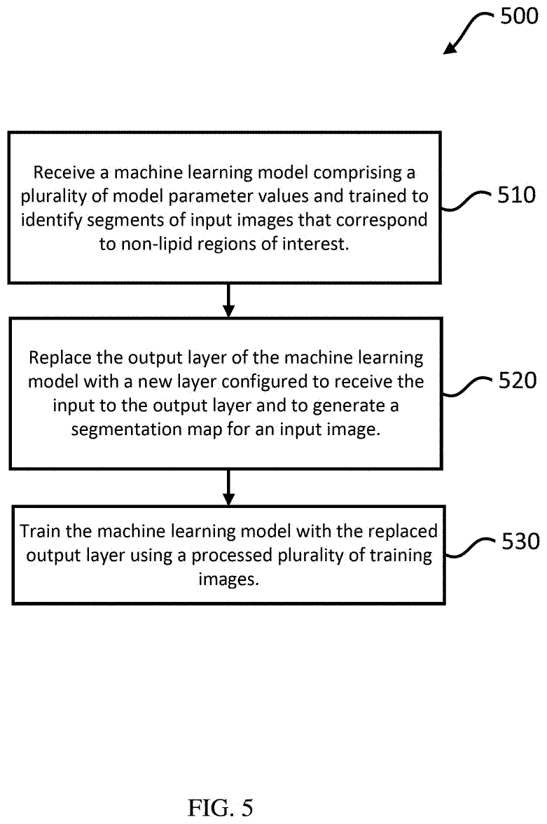

As described in more detail with respect to FIG. 5, existing models can be augmented with existing machine learning models trained to predict non-lipid regions of interest, to also predict the locations of fibrotic caps of lipid pools, as one of a plurality of different output channels. As also described herein with reference to FIGS. 1-2, the fibrotic cap detection model can be trained to output multiple channels each corresponding to a respective region of interest, which can be expressed for example as one or more output images and/or as a segmentation map or other data characterizing regions of an input image as corresponding to different regions of interest, both lipid and non-lipid.

By including additional annotations of non-lipid regions of interest, the fibrotic cap detection model can leverage additional information from more readily discernible regions, such as regions of calcium, to identify fibrotic caps of lipid pools more accurately. This is at least because different regions of interest, such as regions of calcium versus lipid, have different physical characteristics that can be compared within the same image annotated with both types of plaque. As described in more detail, herein, with reference to FIGS. 7-10, the rate of decay of an OCT or other imaging signal, e g , imaging signals generated using IVUS, NIRS, OCT-NIRS, μOCT, etc., through different fibrotic caps can be analyzed by the system to predict fibrotic caps of pools of lipid versus caps of other regions of non-lipid plaque, like calcium. In addition, the fibrotic cap detection model, due to the nature of processing image data annotated with non-lipid regions of interest, can additionally output a segmentation map having multiple channels, allowing more data for analysis to be generated together as opposed to separately.

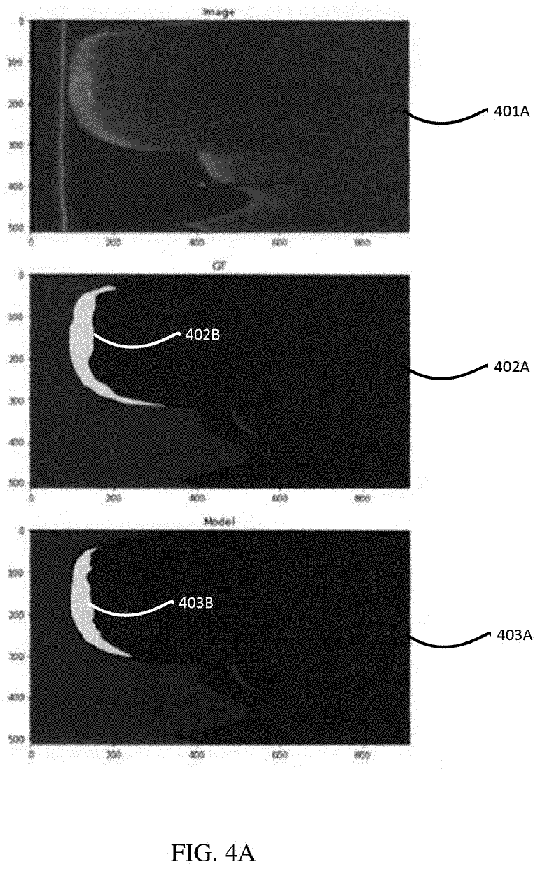

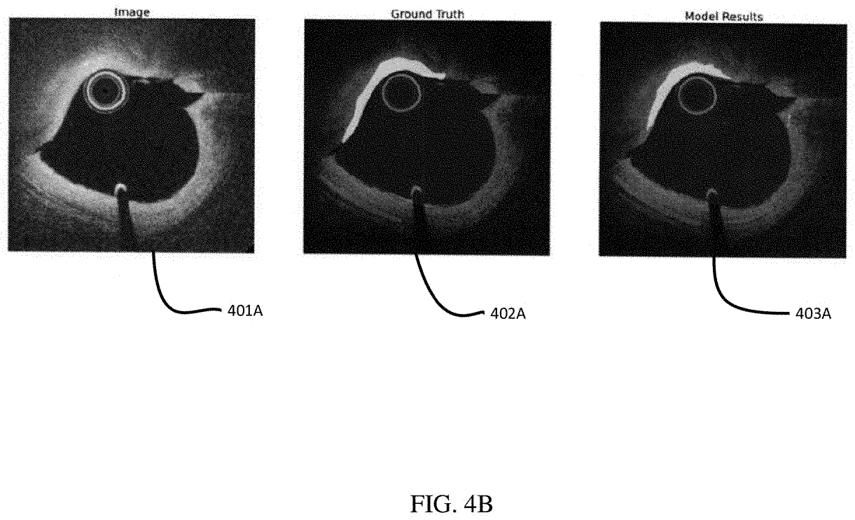

FIG. 4A shows an example input image 401A and corresponding output image 403A generated by the image segmentation system and expressed in polar coordinates, according to aspects of the disclosure. FIG. 4 also shows an image 402A with a ground-truth annotation 402B of a region of a fibrotic cap of a pool of lipid. The image 403A is shown with a model-generated annotation 403B, generated for example using a model trained as described herein with reference to FIG. 3. The images 401A-403A are shown expressed in polar coordinates relative to a reference point, e.g., the center of the lumen for an imaged blood vessel. In some examples, the system can post-process the output image, for example by applying a MAX filter with a kernel size of (15,2). Other types of filters with different sizes may also be used alternatively or in combination. One reason for post-processing can be to smooth the boundary of the annotation before the annotation is displayed.

FIG. 4B shows the example input image 401A and corresponding output image 403A of FIG. 4A expressed in Cartesian coordinates. The image 402A is also shown. FIGS. 4A-B show that the system can be configured to output images in a variety of different formats and according to different coordinate systems.

FIG. 5 is a flowchart of an example process 500 for training a fibrotic cap detection model using a previously trained model, according to aspects of the disclosure. In some examples, the process 500 may be performed as part of a transfer learning procedure, for fine-tuning or training a fibrotic cap detection model using another machine learning model trained to perform a different task.

The system receives a machine learning model, according to block 510. The machine learning model can include a plurality of model parameter values and be trained to identify input images that correspond to non-lipid regions of interest, such as calcium, a lumen, or media in an image of a blood vessel.

The system replaces the output layer of the machine learning model with a new layer configured to receive the input to the output layer and to generate a segmentation map for an input image, according to block 520. For example, the previous output layer can be replaced with a new neural network layer configured to receive input from the previous output layer and output a segmentation map with one or more channels. Initially, the new neural network layer can include randomly initialized model parameter values.

The system trains the machine learning model with the new output layer using a processed plurality of training images. The training images can be processed according to block 320 of the process 300 in FIG. 3 and can each include annotations of locations of non-lipid regions of interest. During training, the system can freeze model parameter values for each layer in the machine learning model except for the new output layer. The system can train the partially frozen model according to any technique for supervised learning, for example using the techniques and training image data described herein with reference to FIG. 3. The system can be configured to train the machine learning model for a certain number of epochs, for example fifty epochs, and then save the best set of model parameter values for the machine learning model identified after processing a validation set of training images through the machine learning model.

The system can load the best model parameter values for the new output layer, e.g., the model parameter values that caused the least amount of loss for a loss function and over a validation set of training images, and train until meeting another one or more training criteria, e.g., 200 epochs, but this time after unfreezing the rest of the model parameter values of the model. In other words, the machine learning model can be trained, and its model parameter values can be updated, for example according to the process 300 described with reference to FIG. 3.

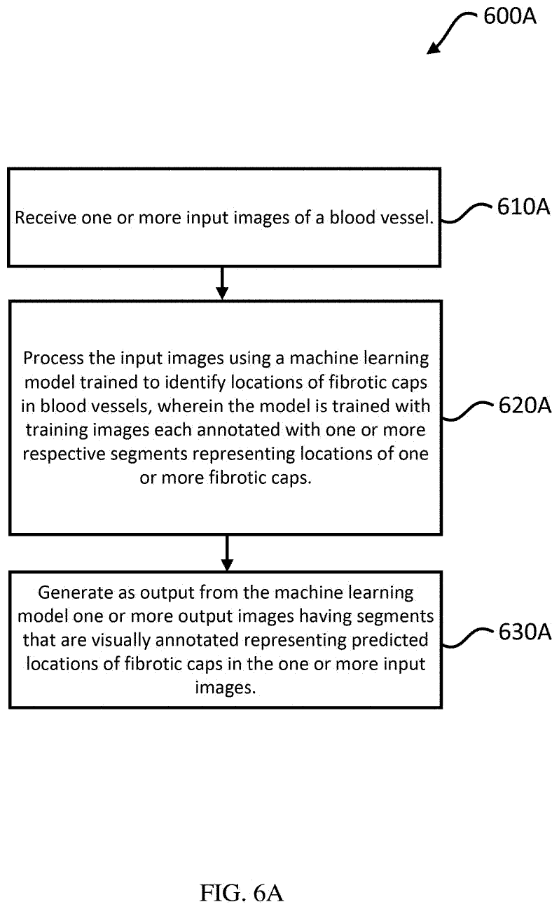

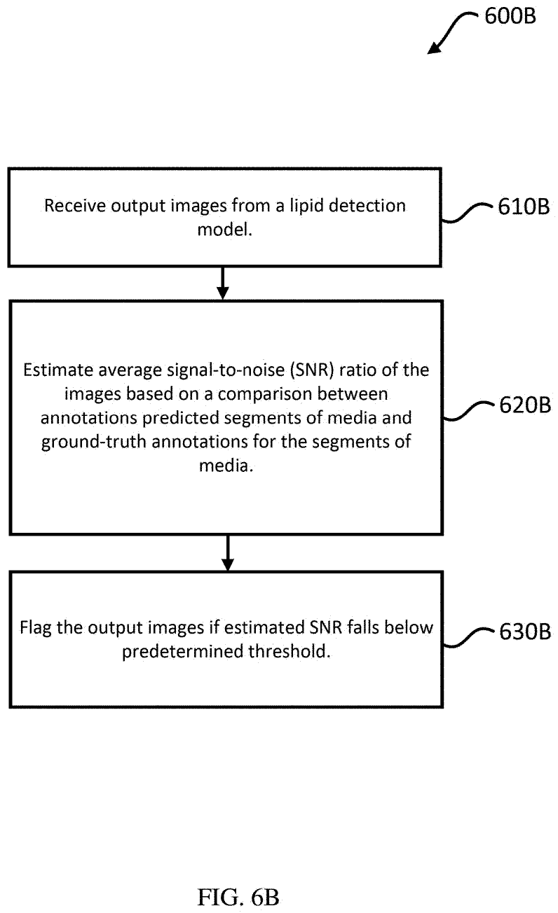

FIG. 6A is a flowchart of an example process 600A for detecting fibrotic caps of lipid pools in tissue surrounding blood vessels, according to aspects of the disclosure.

The system receives one or more input images of a blood vessel, according to block 610. For example, as described herein with reference to FIG. 1, the system can receive the input image 120 and/or one or more additional images.

The system processes one or more input images using a fibrotic cap detection model trained to identify locations of fibrotic caps, according to block 620. The fibrotic cap detection model can be trained with training images each annotated with locations of one or more fibrotic caps. In some examples, the training images are further annotated with locations of non-lipid regions of interest different from the fibrotic caps, such as calcium, media, and the lumen of the blood vessel. The annotations can include a visual boundary overlaid on the output image, or for example provided as separate data, e.g., as a mask of pixels.

In this specification, identification and annotation can be approximative, e.g., within a predetermined margin of error or threshold. An approximative annotation may under-annotate or over-annotate a fibrotic cap within the predetermined margin of error or threshold. Similarly, the identification of a fibrotic cap according to aspects of the disclosure may under-identify or over-identify portions of the input image as corresponding to the identified fibrotic cap, within the predetermined margin of error or threshold.

In some implementations, the system is configured to use predictions of non-lipid segments of an output image as part of estimating the signal-to-noise ratio of a sequence of output images. As described herein, at least because of the physical characteristics of regions of lipid versus regions of non-lipid when interacting with an imaging signal, regions of non-lipids, such as calcium or media, can be identified with higher accuracy versus regions of lipid. While the system can use annotations of regions of non-lipids to predict the locations of fibrotic caps adjoining lipid pools more accurately, as described herein, the system can also leverage the reliability of identifying regions of non-lipid to estimate the signal-to-noise ratio for a sequence of output images.

It has been observed that a lower signal-to-noise ratio (SNR) in a sequence of image frames can correspond to reduced accuracy in identifying and characterizing fibrotic caps in the sequence. The system can estimate the SNR by comparing ground-truth annotations versus predicted annotations of locations in imaged blood vessels corresponding to regions of non-lipid, such as media. If the estimated SNR is low, e.g., below a threshold, the system can flag the sequence as having potentially reduced accuracy in detecting fibrotic caps of lipid pools.

FIG. 6B is a flowchart of an example process for flagging output images of a fibrotic cap detection model with a low signal-to-noise ratio, according to aspects of the disclosure.

The system receives output images from a fibrotic cap detection model, according to block 610B. The output images can be generated, for example, by a fibrotic cap detection model trained as described herein with reference to FIGS. 3 and 5. The output images can correspond to a sequence of input images, for example captured by an imaging device during a pullback as part of an imaging procedure.

In addition to detecting the location of fibrotic caps of lipid pools, the fibrotic cap detection model as described in the process 600B is also trained to detect at least one type of non-lipid region in input images, such as media or calcium. The description that follows describes the use of regions of media in estimating the SNR, although other types of plaque or tissue can be used, such as calcium or background.

The system estimates an average signal-to-noise ratio of the images based on a comparison between annotations of predicted regions of media in the output images, with the ground-truth annotations of the regions of media. The system can receive the ground-truth annotations, for example, as part of a validation set for the output images, or from another source configured to identify and characterize regions of interest, for example the annotation engine 110 of the system 100 of FIG. 1.

The system can estimate noise in an image by computing the standard deviation between a region of the image that should have zero signal, e.g., the lumen of a blood vessel, with the actual value of the signal at the region in the image frame. The standard deviation between the actual and expected value of the signal at that region can be an estimated noise value for the image. Other regions of the image can be selected, in addition to or as an alternative to a region corresponding to the lumen of the imaged blood vessel. For example, the region can be a point far away from the position of the catheter as shown in the image, for example because a region at a point far away enough would register a signal value of zero without noise. Another example region can be the space behind the guidewire of a catheter, as the guidewire would block all signals behind it.

In some implementations, the system can estimate noise by measuring the highest un-normalized intensity from the refraction of tissue depicted in the image frame, ignoring refraction from wires, stents, and catheters. In some examples, the system can estimate SNR by fitting a two-mode Gaussian mixture model to an intensity histogram of the image frame, excluding signal intensity at regions depicting wires, stents, or catheters. The system can divide averages of the two mixture models, where the lower-intensity model would represent noise and the high-intensity model would represent signal. In some examples, the system can estimate SNR by dividing the brightest tissue pixel with the darkest lumen pixel.

The system flags the output images if the estimated SNR is below a predetermined threshold, according to block 630B. The flagged output images can then be set aside and/or manually reviewed, for example. In some examples, the system only performs the process 600B as part of processing a validation set for training the fibrotic cap detection model.

The image segmentation system 100 can be configured to identify and/or characterize fibrotic caps for lipid pools by measuring the radial signal intensity over arc-lines in an input image of a blood vessel. As described herein, different tissues and plaques have different physical characteristics. Example characteristics include the peak intensity and rate of decay for an imaging signal, such as an OCT signal, an IVUS signal, a NIRS signal, an OCT-NIRS signal, a μOCT signal, etc., passing through the tissue or plaque.

The rate of decay for a signal refers to the change of signal strength over increasing distance relative to a reference point. A reference point can be, for example, the center of the lumen of an imaged blood vessel when viewed as a two-dimensional cross-section, or the center of a catheter having an imaging probe from which the signal originally emanates. As the signal propagates through the lumen, tissue, and/or plaque, the signal grows weaker following a peak signal intensity generally occurring at or near the edge of the lumen, for example when the edge of the lumen has a fibrotic cap. The peak signal intensity can occur, for example, as a result of the signal reflecting at least partially off of the edge of the lumen. Past the lumen edge, the signal generally decays until the signal strength is zero or low enough that it can no longer be detected.

As also described herein, one challenge with accurately identifying and characterizing lipid in tissue surrounding a blood vessel stems from lipid's characteristic to quickly decay a penetrating signal. However, the rate of decay has been observed to be relatively consistent across different images of different lipid pools and fibrotic caps across multiple blood vessels. Aspects of the disclosure provide for techniques for identifying a rate of signal decay and comparing that rate to known rates for different tissues and plaques, to identify depicted fibrotic caps in an input image frame.

FIG. 7A illustrates multiple arc-lines 750A-B from the center 760 of a lumen depicted in an image frame 700A. The image frame 700A also depicts the center 770 of an imaging probe from which an imaging signal to capture the image frame 700A originated. The arc-lines 750A-B form a coverage angle corresponding to the portion of the lumen wall occupied by fibrotic cap 790. The arc-lines 750A-B are shown relative to the center 760 of the lumen, as an example, but the arc-lines 750A-B can be relative to any reference point, for example center 770 of a catheter having an imaging probe for capturing the image frame 700A. The system can be configured to measure signal intensity in the image frame along different points of the arc-lines 750A-B.

FIG. 7A depicts the arc-lines 750A-B for illustrative purposes, but the system does not render or draw the arc-lines for display as part of performing the process 700B. In some implementations, the system can be configured to additionally send data for display corresponding to one or more measured arc-lines and their corresponding signal strengths. FIG. 7A also depicts a boundary 780 between a fibrotic cap 785 and a region of lipid 795. As described herein with reference to FIGS. 7B-9, the image segmentation system can be configured to identify fibrotic caps and identify a boundary between the fibrotic cap and the pool of lipid. In some examples, the image segmentation system is configured to receive an input image with a fibrotic cap annotation and refine the annotation to represent a more accurate boundary between the annotated cap and an adjacent pool of lipid.

FIG. 7B is a flowchart of an example process 700B for identifying fibrotic caps based on radial signal intensities for arc-lines in an input image of a blood vessel, according to aspects of the disclosure.

The system receives an input image frame of a blood vessel, according to block 710B. For example, the input image frame can be an image frame received by a user computing device through an imaging device, as described herein with reference to FIG. 1. The input image can include one or more annotations of fibrotic caps surrounding the lumen of the blood vessel. The annotations can be, for example, hand-labeled, or generated by the image segmentation system, as described herein with reference to FIG. 1. In some examples, if the input image is annotated with a fibrotic cap but not arc-lines corresponding to a coverage angle for the cap, the system can be configured to calculate the arc-lines relative to a reference point, such as the center of a lumen depicted in the image frame. As part of generating the arc-lines, the system can identify the center of the lumen.

In some examples, the system receives an input image frame annotated only with arc-lines for one or more fibrotic caps depicted in the input image. In those examples, the input image frame can be annotated by hand, or using one or more machine learning models trained to predict arc-lines defined by a coverage angle for a fibrotic cap depicted in the input image frame.

The system calculates, for each arc-line relative to the center of the lumen of the blood vessel depicted in the image frame, a respective radial signal intensity, according to block 720B. A radial signal intensity (or “signal intensity”) refers to the strength of an imaging signal at a region of the image frame corresponding to a respective arc defined relative to a reference point, such as the center of the lumen of the imaged blood vessel. The system can measure the radial signal intensity along a number of points on each arc-line, with varying distances relative to the reference point.

The system can convert the signal into numerical values for each pixel of an input image frame. For each pixel, the respective numerical value can correspond to the amount of light reflected from the imaging probe at that point. In some examples, the system can normalize each image frame, such that the highest value is 1 and the lowest value is 0. The normalized values within a pixel neighborhood can be averaged to provide a less noisy signal. The pixel neighborhood can be all pixels adjacent to a target pixel, as an example, but the pixel neighborhood can be defined over other pixels relative to a target pixel, from implementation-to-implementation.

The signal intensity measurements for each arc-line can be smoothed to remove noise from the different collected measurements. For example, multiple samples, e.g., 12 samples at a time, can be averaged in the angle dimension (e.g., the angle dimension formed by a line intersecting a point and the reference point, relative to a common origin). As another example, the system can apply a Bartlett or triangular window function, for example with a 13-pixel window size, to reduce noise along the radial dimension (e.g., the radial dimension of a point along an arc-line expressed in polar coordinates) of the endpoints. The signal intensity measurements for each arc-line can be normalized by dividing each value by the signal value at the edge of the lumen.

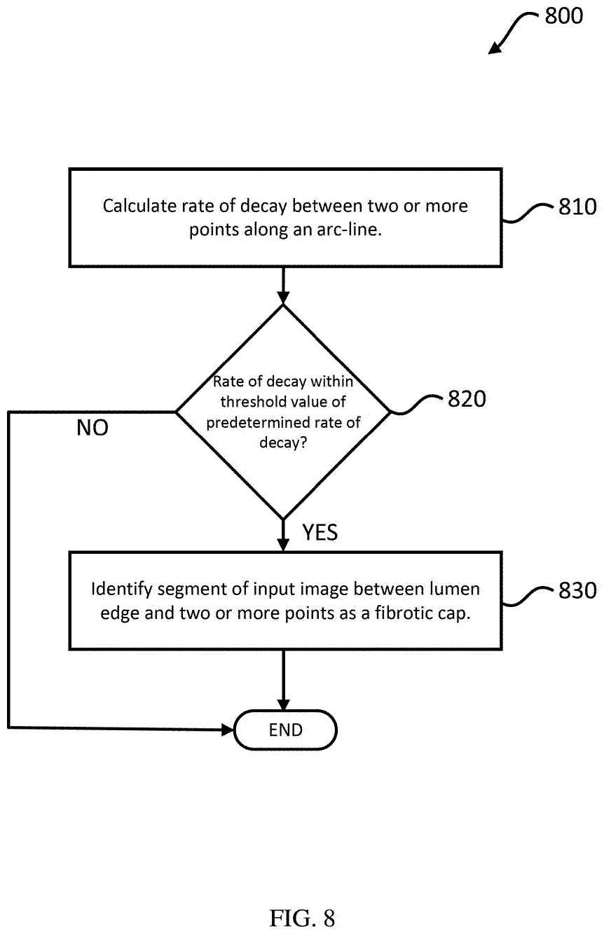

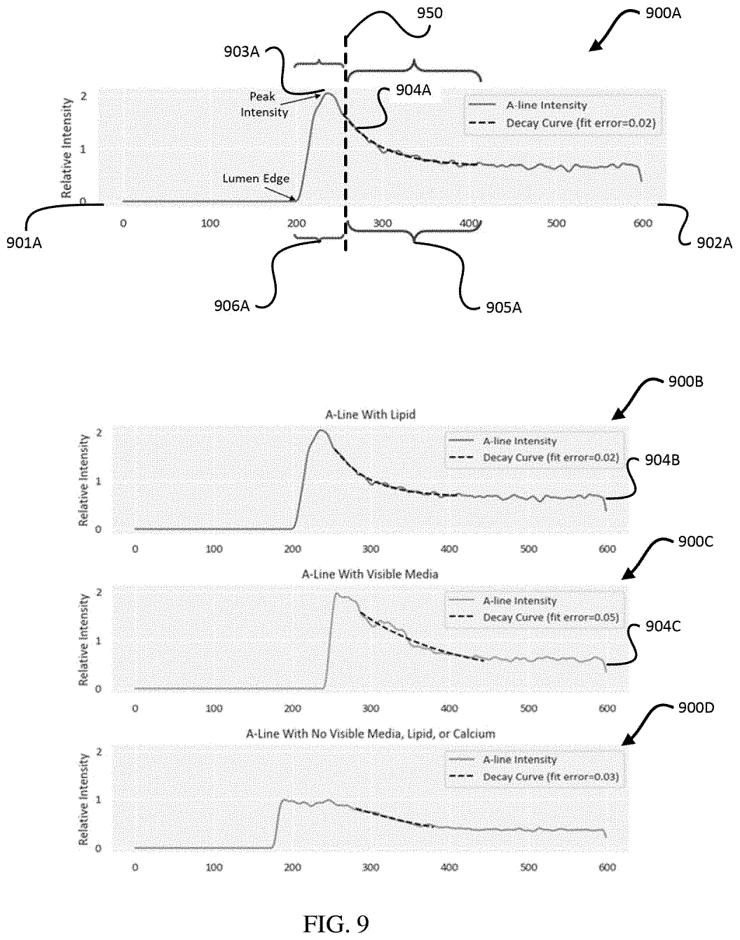

The system identifies, from a plurality of radial signal intensities for points along the arc-lines, one or more fibrotic caps depicted in the input image, according to block 730B. As described herein with reference to FIGS. 8 and 9, the system calculates the rate of decay of the plurality of radial signal intensities and compares that rate to other known rates of decay through different tissues or plaques. For example, arc-lines through lipid have been found to have high signal intensity after the edge of a lumen, with the intensity for subsequent arc-lines receding according to a decay curve, which for example can be exponential over the distance from the reference point.

As part of identifying the fibrotic caps, the system can receive an image annotated with a fibrotic cap and identify a boundary between the fibrotic cap and an adjacent pool of lipid. The system can then update the annotation of the fibrotic cap to reflect the boundary more accurately between the cap and pool. The system can be configured to identify a relative radial intensity for a point along an arc-line as a point corresponding to the boundary. The relative radial intensity can be in proportion to a peak radial intensity measured for another point along the arc-line and corresponding to a wall of the lumen. The relative signal intensity corresponding to the boundary between the fibrotic cap and the adjacent pool of lipid can be based on a decay curve of signal intensity for known samples of images annotated with fibrotic caps, as described below.

In some examples, the final boundary of an initially-generated boundary can be determined by the system by taking the average locations of pixels between the boundaries of annotated fibrotic caps identified in different output images, obtained for example using both the fibrotic cap detection model and using radial intensity analysis, as described herein. The average locations over each of the pixels can form part of the updated boundary.

In some examples, the system can determine the degree of overlap in pixels annotated between separately generated output images and include pixels in the updated boundary that represent the locations at which the separately annotated output images overlap. The system can interpolate the remaining pixels of the updated boundary, e.g., at locations in which the separately generated output images do not overlap in annotation.

In some examples, the system can be configured to first identify fibrotic caps based on the rate of decay of radial signal intensities for a plurality of arc-lines measured from an input image of a blood vessel, and then update the boundary of an annotation of the fibrotic caps using a fibrotic cap detection model. In this way, either approach implemented can be augmented through additional processing by performing the complementary technique described herein.

In some examples, either approach, e.g., using the fibrotic cap detection model or using radial intensity analysis as described herein, can be used to detect false positives or conflicting identifications generated by either approach for the same input image. For example, if an input image is processed by a fibrotic cap detection model, the same input image can also be processed for identifying fibrotic caps based on the rate of decay of radial signal intensities for different arc-lines measured from the input image, for example by performing the processes 700 and 800 described with reference to FIGS. 7 and 8, respectively.

If the output images from the different approaches do not coincide, e.g., exactly or within a predetermined threshold of similarity between annotated output images, then the system can perform one or more actions. For example, the system can flag the disparity for further review, for example by a user. In addition, or alternatively, the system can suggest or automatically select one of the two generated output images to be the “true” output of the system. The system can decide, for example, based on a predetermined preference. In some examples, generating two or more generated output images for the same input using different approaches as described herein can be a user-enabled or -disabled feature for optionally providing error-checking and output validation.

FIG. 8 is a flowchart of an example process 800 of identifying fibrotic caps based on the rate of decay of radial signal intensities for a plurality of arc-lines measured from an input image of a blood vessel.

The system calculates the rate of decay along two or more points of an arc-line, according to block 810. For example, the system calculates the rate of change in signal intensity from different points of arc-lines emanating from the reference point and growing outward and away from the reference point. The system can plot a curve for the rate of decay over the two or more points as a function of distance from the reference point, as shown in FIG. 9, described herein.

The two or more points can be a subset of the plurality of points that are selected based on the distance of the two or more arc-lines relative to the reference point. For example, based on different observations for the likely position from which the rate of decay for signal intensity begins to recede, the system can calculate the rate of decay of arc-lines beginning at that position. The position can be, for example, 1 millimeter from the reference point, although the position can be adjusted closer or farther from the reference point. In some examples, the curve for signal intensity can represent signal intensity over all of the arc-lines measured. In those examples, the rate of decay measured begins with the first of the two or more points closest to the reference point and ends with the last of the two or more points farthest from the reference point.

The system determines whether the rate of decay is within a threshold value of a predetermined rate of decay, according to block 820. For example, the system determines whether the rate of decay is within a threshold value of a predetermined rate of decay by fitting the measured curve for the rate of decay against a known curve, for example a known curve for the rate of decay in signal intensity of arc-lines propagated through a fibrotic cap and a lipid pool. The system can compute the difference or error between the curves using any statistical error-measuring technique, such as the root-mean-square error (RMSE). The RMSE or other technique can generate an error value that the system compares against a predetermined threshold value, which can be for example 0.02. The predetermined threshold value can vary from implementation-to-implementation, for example based on an acceptable tolerance for error in fitting a measured curve to a known curve.

As an example of receiving the known curve, in the context of a fibrotic cap of a pool of lipid, the system can compare the measured curve against a curve calculated over a sample set of images labeled with one or more fibrotic caps of lipid pools. For example, the sample set can include the training image data described herein with reference to FIG. 1, and/or expert-annotated images from one or more other sources. In some examples, the sample set can include image frames annotated by a fibrotic cap detection model trained to predict fibrotic caps in input image frames, as described herein with reference to FIGS. 1-6.

If the system does not determine that the rate of decay is within a threshold value of the predetermined rate of decay (“NO”), then the process 800 ends. Otherwise, the system identifies the segment of the input frame between the two or more points and the edge of the lumen as depicting a fibrotic cap, according to block 830.

In the context of identifying fibrotic caps of lipid pools, the rate of decay corresponds to attenuation of signal strength as the signal passes through the lipid pool. The first point of the two or more points from which the rate of decay was identified can represent the first point at which the signal passes through the pool of lipid. The system can identify the space between the point at the lumen-edge and the point at the boundary of the lipid (indicated by the first of the two or more points at which the compared rate of decay begins in the curve) as the location of a fibrotic cap. By identifying the location of the fibrotic cap, the system also determines the boundary between the cap and an adjacent pool of lipid.

The system can be configured to identify the edge or perimeter of the lumen, for example using the fibrotic cap detection model as described herein and trained to output a channel corresponding to the lumen of an imaged blood vessel.

In some examples, the system can refine training data for training the fibrotic cap detection model, by processing training data to determine whether each image in the training data depicts a fibrotic cap using a radial intensity analysis as described herein. The system can sample some or all of received training data received for training the fibrotic cap detection model and determine whether the sampled training data includes images that do not depict fibrotic caps, within a predetermined measure of confidence. The system can flag these images for further review, for example by manual inspection, to determine whether the images should be discarded or should remain in the training data. In some examples, the system can be configured to perform the flagging and removal automatically.

In some examples, the system can pre-label training data for training the fibrotic cap detection model, by processing training data using a radial intensity analysis as described herein. The pre-labels can be used as labels for the training data and for training the fibrotic cap detection model. In other examples, the pre-labels can be provided for manual inspection, to facilitate the manual labeling of training data with fibrotic caps. In some examples, the system provides at least some of the pre-labels as labels for received training data, and at least some other pre-labels for manual inspection.

In some examples, the system receives an input image frame annotated only with a coverage angle of a corresponding fibrotic cap. From the coverage angle, the system identifies arc-lines containing the fibrotic cap at the annotated angle and can identify the fibrotic cap, as described herein with reference to FIG. 8. The image data can be provided as additional training data for training the fibrotic cap detection model as described herein.

Manual annotation by coverage angle can be easier than annotating the location of the fibrotic cap itself, which can allow for more training data to be generated in the same amount of time. The system can be trained on more data, which can allow for a wider variety of training data to be used for training the system. In addition, generating training data from images annotated with a coverage angle for a fibrotic cap can improve manual annotation by standardizing the annotations across the training data. For example, manual annotators may be more consistent in annotation among one another while annotating for a coverage angle, as opposed to annotating the location of the fibrotic cap itself. The latter may be more susceptible to variability, e.g., different annotators may estimate different thicknesses for the same fibrotic cap.

FIG. 9 shows graphs 900A-D of peak signal intensities and rates of decay through different tissues and plaques. Graph 900A plots relative signal intensity 901A (relative to the highest and lowest detected signal intensity) and distance from reference point 902A, for example, measured in pixels. Solid curve 903A represents the curve measured from a sample set of image frames sharing a common characteristic, for example all depicting fibrotic caps of lipid pools. Dotted curve 904A represents at least a portion of a curve computed from measured arc-lines of an input image frame, for example the two or more arc-lines as described herein with reference to FIG. 900.

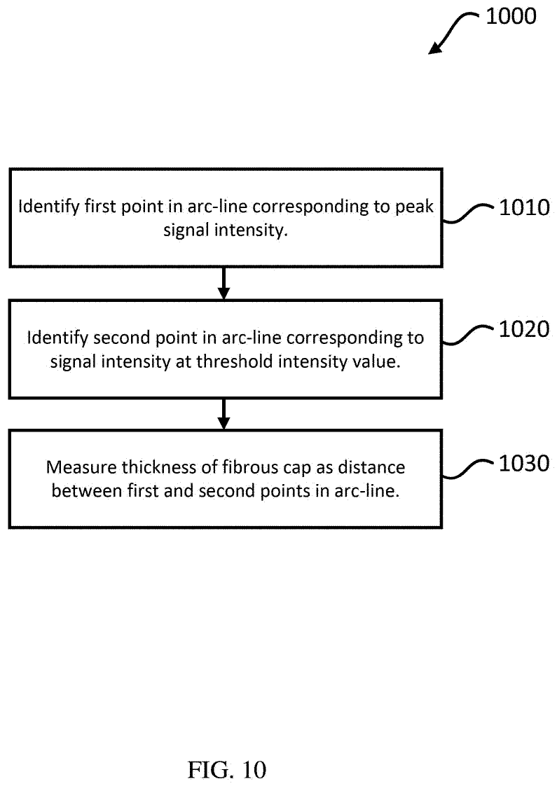

Region 905A corresponds to the region of the image frame in which the decay rate is measured from the two or more points of an arc-line, corresponding to locations within the region. As described herein, the decay rate for the two or more points are fit to the solid curve 903A previously received by the system. In this example, the fit between the curves has an error of 0.02. Region 906A corresponds to the region of the image frame predicted to depict a fibrotic cap for a lipid pool. As described herein with reference to FIG. 10, the system can estimate a thickness for the fibrotic cap of a lipid pool. Line 950 corresponds to the end of the region 906A, which also represents the boundary between the fibrotic cap and the pool of lipid. Put another way, the line 950 corresponds to a point at which the curve 903A begins to decay at the rate corresponding to signal intensity decay previously measured when passing through lipid.

Graphs 900B-D illustrate the dotted curve 904A corresponding to the measured decay rate with solid curves 904B-D previously generated from sets of image frames with different characteristics, e.g., different plaques around imaged blood vessels. Solid curve 904B of graph 900B is a curve measured from a set of image frames depicting fibrotic caps of lipid pools, as described with reference to the graph 900A. The solid curve 904B shows the signal intensity is typically higher (brighter) following the lumen edge.

Solid curve 904C of the graph 900C is a curve measured from a set of image frames depicting visible media in tissue of the imaged blood vessel. The solid curve 904C shows the signal intensity as typically higher (brighter) following the lumen edge, but the rate of decay for the curve 904C does not fit the dotted curve 904A as well as the solid curve 903A or 904B. In the graph 900C, the error in fit between the curves is measured as 0.05.

Solid curve 904D of the graph 900D is a curve measured from a set of image frames not depicting any visible media, calcium, or lipid. The solid curve 904D shows a peak intensity that is lower relative to the lumen edges of the blood vessels measured to generate the solid curves 904A-C, and the fit error between the curve 904D and the dotted curve 904A is also higher (0.03) than the fit for the dotted curve 904A and the solid curve 903A. In some examples, the predetermined threshold can be generated based on comparing curves of known sample sets, e.g., the solid curves 904A-D, and comparing the difference in errors in fit for the different curves.

FIG. 10 is a flowchart of an example process 1000 for measuring a thickness of a fibrotic cap using the peak radial signal intensity in a sequence of measured arc-lines of an imaged blood vessel, according to aspects of the disclosure.

The system identifies a first point of an arc-line corresponding to the peak radial signal intensity, according to block 1010. For example, the system can plot radial signal intensities for multiple points along an arc-line and identify the point with the highest signal intensity value. As described herein with reference to FIGS. 8-9, the peak signal intensity can occur generally after the edge of the lumen of the imaged blood vessel.

The system identifies a second point of an arc-line corresponding to a radial signal intensity meeting a threshold intensity value relative to the peak radial signal intensity, according to block 1020. For example, the threshold intensity value can be set to 80% of the peak signal intensity. The threshold intensity value can be modified from implementation-to-implementation, for example based on an analysis of a sample set of image frames of annotated fibrotic caps, and comparing the signal intensities of points along an arc-line on either end of the fibrotic cap.

The system measures the thickness of a fibrotic cap as the distance between the first and second points in the arc-line, according to block 1030. The system can repeat the process 1000 for multiple arc-lines originating from the same reference point, as well as one or more lines originating from the same reference point that are between arc-lines defining a coverage angle corresponding to a fibrotic cap. For example, because the fibrotic cap may have different thicknesses at different points, the system can measure the thickness along different lines according to the block 1030 to identify regions in which the thickness of the fibrotic cap is larger or smaller.

After estimating the location and thickness of fibrotic caps in received input image frames, the system can be configured to output data defining the estimations, for example on a display or as part of a downstream process for diagnosis and/or analysis, as described herein with reference to FIGS. 1-2. In some examples, the system can be configured to flag image frames, for example through a prompt on a display or through some visual indicator, with predicted fibrotic caps of lipid pools that are thinner than a threshold thickness. The threshold thickness can be set to flag image frames for potential additional review and analysis, for example because image frames depicting fibrotic caps thinner than the threshold thickness may be indicators of increased risk of plaque rupture, such as TCFA, of the image blood vessel.

### Example Computing Environments:

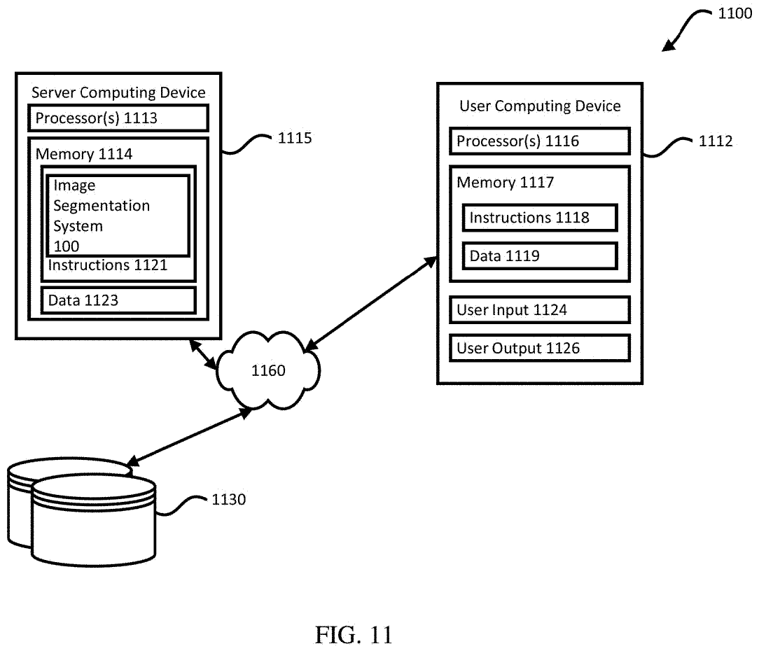

FIG. 11 is a block diagram of an example computing environment implementing the image segmentation system 100, according to aspects of the disclosure. The system 100 can be implemented on one or more devices having one or more processors in one or more locations, such as in server computing device 1115. User computing device 1112 and the server computing device 1115 can be communicatively coupled to one or more storage devices 1130 over a network 1160. The storage device(s) 1130 can be a combination of volatile and non-volatile memory and can be at the same or different physical locations as the computing devices 1112, 1115. For example, the storage device(s) 1130 can include any type of non-transitory computer readable medium capable of storing information, such as a hard-drive, solid state drive, tape drive, optical storage, memory card, ROM, RAM, DVD, CD-ROM, write-capable, and read-only memories.

The server computing device 1115 can include one or more processors 1113 and memory 1114. The memory 1114 can store information accessible by the processor(s) 1113, including instructions 1121 that can be executed by the processor(s) 1113. The memory 1114 can also include data 1123 that can be retrieved, manipulated or stored by the processor(s) 1113. The memory 1114 can be a type of non-transitory computer readable medium capable of storing information accessible by the processor(s) 1113, such as volatile and non-volatile memory. The processor(s) 1113 can include one or more central processing units (CPUs), graphic processing units (GPUs), field-programmable gate arrays (FPGAs), and/or application-specific integrated circuits (ASICs).

The instructions 1121 can include one or more instructions that when executed by the processor(s) 1113, causes the one or more processors to perform actions defined by the instructions. The instructions 1121 can be stored in object code format for direct processing by the processor(s) 1113, or in other formats including interpretable scripts or collections of independent source code modules that are interpreted on demand or compiled in advance. The instructions 1121 can include instructions for implementing the system 100 consistent with aspects of this disclosure. The system 100 can be executed using the processor(s) 1113, and/or using other processors remotely located from the server computing device 1115.

The data 1123 can be retrieved, stored, or modified by the processor(s) 1113 in accordance with the instructions 1121. The data 1123 can be stored in computer registers, in a relational or non-relational database as a table having a plurality of different fields and records, or as JSON, YAML, proto, or XML documents. The data 1123 can also be formatted in a computer-readable format such as, but not limited to, binary values, ASCII or Unicode. Moreover, the data 1123 can include information sufficient to identify relevant information, such as numbers, descriptive text, proprietary codes, pointers, references to data stored in other memories, including other network locations, or information that is used by a function to calculate relevant data.

The user computing device 1112 can also be configured similar to the server computing device 1115, with one or more processors 1116, memory 1117, instructions 1118, and data 1119. The user computing device 1112 can also include a user output 1126, and a user input 1124. The user input 1124 can include any appropriate mechanism or technique for receiving input from a user, such as keyboard, mouse, mechanical actuators, soft actuators, touchscreens, microphones, and sensors.

The server computing device 1115 can be configured to transmit data to the user computing device 1112, and the user computing device 1112 can be configured to display at least a portion of the received data on a display implemented as part of the user output 1126. The user output 1126 can also be used for displaying an interface between the user computing device 1112 and the server computing device 1115. The user output 1126 can alternatively or additionally include one or more speakers, transducers or other audio outputs, a haptic interface or other tactile feedback that provides non-visual and non-audible information to the platform user of the user computing device 1112.

Although FIG. 11 illustrates the processors 1113, 1116 and the memories 1114, 1117 as being within the computing devices 1115, 1112, components described in this specification, including the processors 1113, 1116 and the memories 1114, 1117 can include multiple processors and memories that can operate in different physical locations and not within the same computing device. For example, some of the instructions 1121, 1118 and the data 1123, 1119 can be stored on a removable SD card and others within a read-only computer chip. Some or all of the instructions and data can be stored in a location physically remote from, yet still accessible by, the processors 1113, 1116. Similarly, the processors 1113, 1116 can include a collection of processors that can perform concurrent and/or sequential operation. The computing devices 1115, 1112 can each include one or more internal clocks providing timing information, which can be used for time measurement for operations and programs run by the computing devices 1115, 1112.

The devices 1112, 1115, can be capable of direct and indirect communication over the network 1160. For example, using a network socket, the user computing device 1112 can connect to a service operating in the datacenter 1150 through an Internet protocol. The devices 1115, 1112 can set up listening sockets that may accept an initiating connection for sending and receiving information. The network 1160 itself can include various configurations and protocols including the Internet, World Wide Web, intranets, virtual private networks, wide area networks, local networks, and private networks using communication protocols proprietary to one or more companies. The network 1160 can support a variety of short- and long-range connections. The short- and long-range connections may be made over different bandwidths, such as 2.402 GHz to 2.480 GHz (commonly associated with the Bluetooth® standard), 2.4 GHz and 5 GHz (commonly associated with the Wi-Fi® communication protocol); or with a variety of communication standards, such as the LTE® standard for wireless broadband communication. The network 1160, in addition or alternatively, can also support wired connections between the devices 1112, 1115, including over various types of Ethernet connection.

Although a single server computing device 1115 and user computing device 1112 are shown in FIG. 11, it is understood that the aspects of the disclosure can be implemented according to a variety of different configurations and quantities of computing devices, including in paradigms for sequential or parallel processing, or over a distributed network of multiple devices. In some implementations, aspects of the disclosure can be performed on a single device, and any combination thereof.

While operations shown in the drawings and recited in the claims are shown in a particular order, it is understood that the operations can be performed in different orders than shown, and that some operations can be omitted, performed more than once, and/or be performed in parallel with other operations. Further, the separation of different system components configured for performing different operations should not be understood as requiring the components to be separated. The components, modules, programs, and engines described can be integrated together as a single system or be part of multiple systems. In addition, as described herein, an image segmentation system, such as the image segmentation system 100 of FIG. 1 can perform the processes described herein.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A method for fibrotic cap identification in blood vessels, the method comprising:
receiving, by one or more processors, one or more input images of a blood vessel;
processing, by the one or more processors, the one or more input images using a machine learning model trained to identify locations of fibrotic caps in blood vessels, wherein the machine learning model is trained using a plurality of training images annotated with one or more locations of one or more fibrotic caps, each fibrotic cap adjacent to a respective pool of lipid;
receiving, by the one or more processors and as output from the machine learning model, one or more output images having segments that are visually annotated representing predicted locations of fibrotic caps; and
generating, using the one or more processors and from the one or more output images, an updated boundary of a fibrotic cap relative to an adjacent pool of lipid based on signal intensities for a plurality of points in the one or more input images.

2. The method of claim 1, wherein the one or more input images are further annotated with segments corresponding to locations of at least one of calcium, a lumen in the blood vessel, or media.

3. The method of claim 2,
wherein the one or more input images comprise annotated segments representing one or more regions of media;
wherein the one or more input images are images received from an imaging probe during a pullback of the imaging probe in the blood vessel; and
wherein the method further comprises:
estimating, by the one or more processors, the average signal-to-noise ratio (SNR) of the one or more input images based on comparisons of predicted annotations of regions of media in the one or more input images and one or more ground-truth annotations of regions of media in the one or more input images; and
in response, flagging, by the one or more processors, the one or more output images corresponding to the one or more input images in response to determining that the average SNR falls below a predetermined threshold.

4. The method of claim 3, wherein the imaging probe is an optical coherence tomography (OCT) imaging probe, an intravascular ultrasound (IVUS) imaging probe, a near-infrared spectroscopy (NIRS) imaging probe, an OCT-NIRS imaging probe, or a micro-OCT (μOCT) imaging probe.

5. The method of claim 1, wherein the plurality of points are along one or more arc-lines enclosing the fibrotic cap.

6. The method of claim 1, wherein receiving the one or more output images comprises receiving, for each input image, a respective visually annotated segment of the input image representing a predicted location for a fibrotic cap.

7. The method of claim 1, wherein the method further comprises receiving, by the one or more processors and for each of the one or more output images, one or more measures of thickness for each fibrotic cap whose location is predicted in the output image.

8. The method of claim 1, wherein generating the updated boundary comprises:
measuring, by the one or more processors, signal intensities for a plurality of points along one or more arc-lines enclosing the fibrotic cap; and
determining, by the one or more processors and based on a comparison of a measured rate of decay of the signal intensities for the plurality of points and a predetermined rate of decay of signal intensity through fibrotic caps of lipid, a boundary between the fibrotic cap and the adjacent pool of lipid.

9. The method of claim 8, wherein determining the boundary between the fibrotic cap and the adjacent pool of lipid comprises identifying a point of the plurality of points having a measured signal intensity that is proportional within a predetermined threshold to a peak signal intensity of the plurality of points.

10. A system comprising:
one or more processors configured to:
receive one or more input images of a blood vessel;
process the one or more input images using a machine learning model trained to identify locations of fibrotic caps in blood vessels, wherein the machine learning model is trained using a plurality of training images annotated with locations of one or more fibrotic caps, each fibrotic cap adjacent to a respective pool of lipid;
receive, as output from the machine learning model, one or more output images having segments that are visually annotated representing predicted locations of fibrotic caps; and
generate from the one or more output images, an updated boundary of a fibrotic cap relative to an adjacent pool of lipid based on signal intensities for a plurality of points in the one or more input images.

11. The system of claim 10, wherein the one or more input images are further annotated with segments corresponding to locations of at least one of calcium, a lumen in the blood vessel, or media.

12. The system of claim 11,
wherein the one or more input images comprise annotated segments representing one or more regions of media;
wherein the one or more input images are images received from an imaging probe during a pullback of the imaging probe in the blood vessel; and
wherein the one or more processors are further configured to:
estimate the average signal-to-noise ratio (SNR) of the one or more input images based on comparisons of predicted annotations of regions of media in the one or more input images and one or more ground-truth annotations of regions of media in the one or more input images; and
in response, flag the one or more output images corresponding to the one or more input images in response to determining that the average SNR falls below a predetermined threshold.

13. The system of claim 12, wherein the imaging probe is an optical coherence tomography (OCT) imaging probe, an intravascular ultrasound (IVUS) imaging probe, a near-infrared spectroscopy (NIRS) imaging probe, an OCT-NIRS imaging probe, or a micro-OCT (μOCT) imaging probe.

14. The system of claim 10, wherein the plurality of points are along one or more arc-lines enclosing the fibrotic cap.

15. The system of claim 11, wherein receiving the one or more output images comprises receiving, for each input image, a respective visually annotated segment of the input image representing a predicted location for a fibrotic cap.

16. The system of claim 11, wherein the one or more processors are further configured to receive, for each of the one or more output images, one or more measures of thickness for each fibrotic cap whose location is predicted in the output image.

17. The system of claim 11,
wherein the system further comprises an imaging probe communicatively connected to the one or more processors; and
to receive the one or more input images of the blood vessel, the one or more processors are further configured to receive image data corresponding to the one or more input images from the imaging probe while the imaging probe is inside the blood vessel.

18. The system of claim 11, wherein in generating the updated boundary, the one or more processors are further configured to:
measure signal intensities for a plurality of points along one or more arc-lines enclosing the fibrotic cap; and
determine, based on a comparison of a measured rate of decay of the signal intensities for the plurality of points and a predetermined rate of decay of signal intensity through fibrotic caps of lipid, a boundary between the fibrotic cap and the adjacent pool of lipid.

19. The system of claim 18, wherein to determine the boundary between the fibrotic cap and the adjacent pool of lipid, the one or more processors are further configured to identify a point of the plurality of points having a measured signal intensity that is proportional within a predetermined threshold to a peak signal intensity of the plurality of points.

20. One or more non-transitory computer-readable media storing instructions that when executed by one or more processors causes the one or more processors to perform operations comprising:
receiving one or more input images of a blood vessel;
processing the one or more input images using a machine learning model trained to identify locations of fibrotic caps in blood vessels, wherein the machine learning model is trained using a plurality of training images each annotated with locations of one or more fibrotic caps, each fibrotic cap adjacent to a respective pool of lipid;
receiving, as output from the machine learning model, one or more output images having segments that are visually annotated representing predicted locations of fibrotic caps generating from the one or more output images, an updated boundary of a fibrotic cap relative to an adjacent pool of lipid based on signal intensities for a plurality of points in the one or more input images.
